(19) United States
(12) Patent Application Publication
(10) Pub. No.: US 2022/0332836 A1
(43) Pub. Date: Oct. 20, 2022

(54) A METHOD FOR PREVENTING HUMAN VIRUS ASSOCIATED DISORDERS IN PATIENTS

(71) Applicant: NOVARTIS AG, Basel (CH)

(72) Inventors: TINA RUBIC-SCHNEIDER, Basel (CH); Elisabetta TRAGGIA, Basel (CH); Peter ULRICH, Schopfheim (DE)

(21) Appl. No.: 17/640,942

(22) PCT Filed: Sep. 11, 2019
(86) PCT No.: PCT/IB2019/057658

§ 371 Date: Mar. 7, 2022

(51) Int. Cl. C07K 16/28; A61K 31/436; A61K 39/395

(52) U.S. Cl. CPC C07K 16/2878; A61K 31/436; A61K 39/3955; C07K 2317/92; A61K 2039/505

(57) ABSTRACT

The disclosure relates to methods of preventing an human virus-associated disorder, in particular EBV-associated disorder, as well as to a method of controlling EBV load in a subject, in particular in a subject being at risk of developing such a disorder like solid organ transplantation patients.

### FIELD OF THE INVENTION

The invention relates to methods for preventing virus associated disorders in an individual at risk of an infection.

### BACKGROUND OF THE INVENTION

The Epstein-Barr virus (EBV) is one of the most successful pathogens in humans with more than 90% of the adult population persistently infected (Cesarman 2014, Cohen 2015). EBV infection in immunodeficient individuals can be associated with all sorts of diseases, including malignancies, including carcinoma (gastric or nasopharyngeal) or lymphoma (e.g. Hodgkin lymphoma). For patients with end-stage organ failure transplantation is the treatment of choice. But success of the transplantation depends on immunosuppressive drugs to prevent organ rejection reactions. EBV-associated post-transplant lymphoproliferative disorders (PTLD) is linked to EBV primary infection or reactivation. In an immunocompetent individual, the anti-viral T cell response controls the infection, but EBV remains latent in memory B cells and some other cell types. In transplanted patients, current immunosuppression therapies dampen the anti-EBV T cell response, leaving EBV-induced B cell proliferation uncontrolled. EBV transplant patients, in particular EBV naive patients, who receive organs from EBV seropositive donors are at risk of developing EBV-associated post-transplant lymphoproliferative disorders (PTLD). In addition, EBV-positive recipients have increased risk, albeit small, to develop such serious disorders. Another virus, JC virus, is responsible for the feared complication of Progressive Multifocal Leukoencephalopathy (PML). As for EBV, JC virus infected cell are under anti-viral T-cell control. Current immunosuppressive agents affecting T-cell function, increase the risk for activation of latent viruses as EB and JC. Despite good short-term results after kidney transplantation, the mid-term results are unfortunate with 30% of patients losing their kidney function at 5 years and 50% at 10 years mainly due to calcineurin inhibitors (CNI), like Cyclosporine A (CsA) or Tacrolimus toxicity. One CNI-free regimen on the market is Belatacept (anti-CTLA-4 Ig) given in combination with mycophenolic acid. Belatacept blocks CD80/CD86 and thereby inhibits T cell costimulation and consequently T cell activation (Larsen 2005, Vincenti 2005). The use of Belatacept is contraindicated in EBV seronegative transplant patients, because of the increased posttransplant lymphoproliferative disorder (PTLD) risk (Hardinger et al., International Journal of Nephrology and Renovascular Disease 2016:9 139-150). By replacing (CNI-free) or reducing tacrolimus (CNI-synergy), new regimens could offer standard-of-care efficacy with a better safety profile and better long-term graft survival. Thus, there is an urgent need for novel therapies that modulate, prevent or inhibit virus infections, like EBV or JCV infections and associated disease, in particular preventing or reducing the likelihood of developing PTLD in EBV-positive or EBV-naive transplant patients or PML caused by JC virus.

### SUMMARY OF THE INVENTION

Epstein Barr Virus (EBV)-associated disorders, like post-transplant lymphoproliferative disorders (PTLD) are linked to EBV primary infection or reactivation. In an immunocompetent individual, the anti-viral T cell response controls the infection but EBV remains latent in B cells and some other cell types. In transplanted patients, immunosuppression could dampen the anti-EBV T cell response, leaving EBV-induced B cell proliferation uncontrolled. The inventors have found out that a CD40 antagonist is a suitable treatment for the prevention of an EBV-associated disorder in a subject at risk of developing such a disorder. In an EBV control assay testing Cyclosporine A (CsA), CTLA4-Ig fusion protein (Belatacept) and the anti-CD40 mAb (CFZ533/iscalimab) the inventors have surprisingly found that the CD40 antagonist does not impair EBV control in vitro, in contrast to CsA and Belatacept. Therefore, the use of CD40 antagonist, in particular anti CD40 antibodies like CFZ533/iscalimab, provide the basis for new transplantation methods reducing the risk of EBV-associated post-transplant disorders, like lymphoproliferative disorders (PTLD). The herein disclosed invention provides a solution for a long-standing need of reducing the risk that EBV-naive transplant patients who receive organs from EBV seropositive donors develop EBV-associated diseases like PTLD.

According to a first aspect of the disclosure a method of preventing a human virus associated disorder in a subject at risk of developing such a disorder is provided, comprising administering to said subject a CD40 antagonist.

In one embodiment of the first aspect of the disclosure, the virus is a human herpesvirus like EBV or Cytomegalovirus (CMV) or a betapolyomavirus like John Cunningham virus (JCV).

According to the first aspect of the disclosure a method of preventing an EBV-associated disorder in a subject at risk of developing such a disorder is provided, comprising administering to said subject a CD40 antagonist.

A second aspect of the disclosure relates to a method of transplanting a solid organ to an EBV seronegative patient in need thereof, comprising administering to the subject a CD40 antagonist.

In a third aspect the disclosure relates to a method of controlling EBV infection in a subject, comprising administering to said subject a therapeutically effective dose of a CD40 antagonist.

In one embodiment, the method according to any of the above described first, second or third aspect of the disclosure is a method of reducing the likelihood that a subject will develop an EBV-associated disorder, comprising administering to the subject a CD40 antibody.

In another embodiment, the method according to any of the above-described embodiments according to any of the above described first, second or third aspect of the disclosure, is a method wherein the subject at risk of developing an EBV-associated disorder will undergo an organ or tissue transplantation.

In one embodiment the method according to any of the above-described embodiments of the first, second or third aspect of the disclosure is a method wherein the subject is (i) an EBV-naive seronegative transplant patient who receives an organ from an EBV seropositive donor, or (ii) an EBV naive patient receiving an organ from an EBV naive donor, or (iii) an EBV seropositive patient receiving an organ from an EBV seropositive or seronegative donor.

In an additional embodiment the method according to any of the above-described embodiments of the first, second or third aspect of the disclosure is a method wherein the patient is a pediatric patient. In another embodiment the pediatric patient is a liver transplants patient.

In another embodiment, the method according to any of the above-described embodiments of the first, second or third aspect of the disclosure is a method wherein the subject at risk of developing an EBV-associated disease is immunosuppressed or receiving immunomodulatory drugs.

In one embodiment the method according to any of the above-described embodiments of the first, second or third aspect of the disclosure is a method wherein the EBV-associated disorder is cancer or a lymphoproliferative disease.

In an additional embodiment of the first, second or third aspect of the disclosure, including all the above-described embodiments thereof, the method reduces the likelihood of developing the post-transplant iymphoproliferative disease.

In an additional embodiment the method according to the first, second or third aspect of the disclosure, including all the above-described embodiments thereof, comprises the transplantation of a solid organ from an EBV seropositive donor to an EBV seronegative recipient.

In another embodiment the method according to the first, second or third aspect of the disclosure, including all the above-described embodiments thereof, is a method wherein the subject at risk of developing an EBV-associated disorder will undergo an organ or tissue transplantation.

In one embodiment the method according to the first, second or third aspect of the disclosure, including all the above-described embodiments thereof, is a method wherein the subject is an EBV-naive seronegative transplant patient who receives an organ from an EBV positive donor.

In an additional embodiment the method according to the first, second or third aspect of the disclosure, including all the above-described embodiments thereof, is a method wherein the patient is a pediatric patient. In another embodiment the pediatric patient is a liver transplants patient.

In another embodiment, the method according to the first, second or third aspect of the disclosure, including all the above-described embodiments thereof, is a method wherein the subject is immunosuppressed.

In a fourth aspect of the disclosure the method according to any of the above-described aspects, including all embodiments thereof, is a method wherein the CD40 antagonist is an antibody. In one embodiment the CD40 antibody is ASKP1240 as described e.g. in U.S. Pat. No. 8,568,725B2, BI655064 as described e.g. in U.S. Pat. No. 8,591,900, FFP104 as described e.g. in U.S. Pat. No. 8,669,352, or MED14920.

In one embodiment of the above-described fourth aspect of the disclosure, the anti-CD40 antibody is an antibody with silenced ADCC activity.

In another embodiment, the antibody used in a method according to any of the above-described embodiments of the fourth aspect of the disclosure is selected from the group consisting of:
a. an anti-CD40 antibody comprising an immunoglobulin VH domain comprising the amino acid sequence of SEQ ID NO: 7 and an immunoglobulin VL domain comprising the amino acid sequence of SEQ ID NO: 8;
b. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
c. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13; and
d. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14.

In one embodiment, the antibody for use in the methods according to anyone of the previous aspects, including all embodiments thereof, is selected from the group consisting of an antibody comprising the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

In one aspect of the invention, the antibody used in the methods according to anyone of the previous aspects, including all embodiments thereof, is CFZ533.

In a fifth aspect of the disclosure, a pharmaceutical composition is provided comprising a therapeutically effective amount of a CD40 antibody, e.g. CFZ533 and one or more pharmaceutically acceptable carriers for use in the methods according to anyone of the previous aspects, including all embodiments thereof.

In one embodiment of the fifth aspect of the disclosure, the route of administration of the pharmaceutical composition comprising the CD40 antibody, e.g. CFZ533, is subcutaneous or intravenous or a combination of subcutaneous or intravenous, wherein the dose may be adjusted so that plasma or serum concentration of antibody is at least 40 μg/mL.

In another embodiment according the fifth aspect, including all embodiments thereof, the pharmaceutical composition comprising the CD40 antibody, e.g. CFZ533, is administered at a dose that may be above 3 mg active ingredient per kilogram of human subject (mg/kg), such as above or equal to 10 mg/kg, above or equal to 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg or 30 mg/kg.

In another embodiment according the fifth aspect, including all embodiments thereof, the administered dose of the pharmaceutical composition comprising the CD40 antibody is about 3 mg to about 30 mg active ingredient per kilogram of a human subject, such as about 3 mg to about 30 mg active ingredient per kilogram when administered intravenously (IV).

In an additional embodiment of the fifth aspect, including all embodiments thereof, the administered dose of the pharmaceutical composition comprising the CD40 antibody, e.g. CFZ533, is about 10 mg active ingredient per kilogram of a human subject, such as about 10 mg active ingredient per kilogram IV.

In one embodiment of the fifth aspect, including all embodiments thereof, the administered dose of the pharmaceutical composition comprising the CD40 antibody, e.g.

CFZ533, is about 150 mg to about 600 mg active ingredient, such as about 150 mg to about 600 mg when administered subcutaneously (SC).

In one embodiment of the fifth aspect, including all embodiments thereof, the administered dose of the pharmaceutical composition comprising the CD40 antibody, e.g.

CFZ533, is about 300 mg or about 450 mg active ingredient, such as about 300 mg or about 450 mg SC.

In an additional embodiment of the fifth aspect, including all embodiments thereof, the CD40 antibody, e.g. CFZ533, is administered first through a loading dose regimen followed by a maintenance dose regimen.

In one embodiment of the fifth aspect, including all embodiments thereof, relates to a method wherein the loading dosing of the pharmaceutical composition comprising the CD40 antibody, e.g. CFZ533, consists of one, two, three or four weekly intravenous or subcutaneous injections of a first dose and the maintenance dosing consists of weekly or biweekly subcutaneous injections of a second dose, and wherein the first dose is higher than the second dose.

Another embodiment of the fifth aspect, including all embodiments thereof, relates to a method wherein the first dose of the pharmaceutical composition comprising the CD40 antibody, e.g. CFZ533, is between about 300 mg and about 600 mg, and the second dose is about 300 mg, about 450 mg or about 600 mg.

In one embodiment of the fifth aspect, including all embodiments thereof, the loading dosing of the pharmaceutical composition comprising the CD40 antibody, e.g. CFZ533, consists of one or two intravenous administration of a first dose and the maintenance dosing consists of weekly or biweekly subcutaneous injections of a second dose.

Another embodiment of the fifth aspect, including all embodiments thereof, the first dose of the pharmaceutical composition comprising the CD40 antibody, e.g. CFZ533, is about 10 mg/kg or about 30 mg/kg and the second dose is between about 300 mg and 600 mg.

An additional sixth aspect of the disclosure relates to the use of a liquid pharmaceutical composition comprising an anti-CD40 antibody, for the manufacture of a medicament for preventing an EBV-associated disorder in a subject at risk of developing such a disorder, wherein the anti-CD40 antibody:
a. is to be intravenously or subcutaneously administered with a first loading dose regimen; and
b. thereafter, with a second maintenance dose regimen, wherein the maintenance dose is different from the loading dose, and wherein said anti-CD40 antibody is selected from the group consisting of:
i. an anti-CD40 antibody comprising an immunoglobulin VH domain comprising the amino acid sequence of SEQ ID NO: 7 and an immunoglobulin VL domain comprising the amino acid sequence of SEQ ID NO: 8;
ii. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
iii. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13;
iv. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14;
v. an anti-CD40 antibody comprising a silent Fc IgG1 region: and
vi. an anti-CD40 antibody comprising the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

An additional embodiment of the sixth aspect, including all embodiments thereof, relates to the use of a liquid pharmaceutical composition comprising an anti-CD40 antibody, for the manufacture of a medicament for reducing the likelihood of post-transplant lymphoproliferative disease.

Another embodiment of the sixth aspect, including all embodiments thereof, relates to the use of a liquid pharmaceutical composition comprising an anti-CD40 antibody, for the manufacture of a medicament for use in a method of transplanting a solid organ to an EBV seronegative patient in need thereof. In another embodiment the sixth aspect of the disclosure relates to the use of an anti-CD40 antibody for the manufacture of a medicament for use in a method of transplanting a solid organ from an EBV seropositive donor to an EBV seronegative recipient.

In a seventh aspect the disclosure relates to an anti-CD40 antibody, e.g. CFZ533, for use according to any of the above described aspect, including all embodiment thereof, wherein the anti-CD40 antibody treatment occurs post-transplantation and the antibody is administered so that plasma or serum concentration of the antibody is at least 40 μg/mL. In one embodiment, the disclosure relates to CFZ533 for use according to aspect seven, including all embodiment thereof, wherein the antibody is administered as a dose of about 3 mg to about 30 mg active ingredient per kilogram of a human subject.

In one embodiment the disclosure relates to CFZ533 for use according to aspect seven, including all embodiment thereof, wherein the dose of the antibody is about 10 mg active ingredient per kilogram of the human subject.

In another embodiment the disclosure relates to CFZ533 for use according to aspect seven, including all embodiment thereof, wherein the antibody is administered as a dose of about 150 mg to about 600 mg active ingredient.

In one embodiment of the seventh aspect the disclosure relates to CFZ533 for use according to anyone of the previous aspects, including all embodiments thereof, wherein the dose is about 300 mg, about 450 mg, or about 600 mg active ingredient. Alternatively, the dose is 300 mg, 450 mg, or 600 mg active ingredient

In one embodiment of the seventh, the disclosure relates to CFZ533 for use according to anyone of the previous aspects, including all embodiments thereof, wherein the antibody is administered with a loading dosing and a maintenance dosing.

In one embodiment of the seventh aspect, the disclosure relates to CFZ533 for use in a method according to anyone of the previous aspects, including all embodiments thereof, wherein the loading dosing consists of one, two, three or four weekly subcutaneous injection(s) of a first dose and the maintenance dosing consists of weekly or biweekly subcutaneous injections of a second dose, and wherein the first dose is higher than the second dose.

In another embodiment of the seventh aspect, the disclosure relates to CFZ533 for use in a method according to anyone of the previous aspects, including all embodiments thereof, wherein the first dose is between about 300 mg and about 600 mg and the second dose is about 300 mg, about 450 or about 600 mg. Alternatively, the dose is between 300 mg and 600 mg and the second dose is 300 mg, 450 or 600 mg.

Additionally, in one embodiment of the seventh aspect, the disclosure relates to CFZ533 for use in a method according to anyone of the previous aspects five and six, including all embodiments thereof, wherein the loading dosing consists of one, two, three or four intravenous administration(s) of a first dose and the maintenance dosing consists of weekly subcutaneous injections of a second dose.

In another embodiment of the seventh aspect, the disclosure relates to CFZ533 for use in a method according to anyone of the previous aspects five and six, including all embodiments thereof, wherein the first dose is about 10 mg/kg and the second dose is about 300 mg, about 450 or about 600 mg active ingredient. Alternatively, the first dose is 10 mg/kg and the second dose is 300 mg, 450 or 600 mg active ingredient.

In an eights aspect the disclosure relates to CFZ533 for use according to anyone of the previous aspects, including all embodiments thereof, wherein the solid organ transplantation is kidney transplantation, liver transplantation, heart transplantation, lung transplantation, pancreas transplantation, intestine transplantation, composite tissue transplantation, bone marrow transplantation or allogeneic haematopoietic stem cell transplantation.

According to a ninth aspect of the disclosure a method of preventing an JCV associated disorder in a subject at risk of developing such a disorder is provided, comprising administering to said subject a CD40 antagonist.

A tenth aspect of the disclosure relates to a method of transplanting a solid organ to a JCV seronegative patient in need thereof, comprising administering to the subject a CD40 antagonist.

In an eleventh aspect the disclosure relates to a method of controlling JCV infection in a subject, comprising administering to said subject a therapeutically effective dose of a CD40 antagonist.

In one embodiment, the method according to any of the above described ninth, tenth or eleventh aspect of the disclosure is a method of reducing the likelihood that a subject will develop an JCV associated disorder, comprising administering to the subject a CD40 antibody.

In another embodiment, the method according to any of the above-described embodiments nine, ten or eleven, is a method wherein the subject at risk of developing a JCV associated disorder will undergo an organ or tissue transplantation.

In one embodiment the method according to any of the above-described embodiments of the ninth, tenth or eleventh aspect of the disclosure is a method wherein the subject is an EBV-naive seronegative transplant patient who receives an organ from a JCV positive donor.

In an additional embodiment the method according to any of the above-described embodiments of the ninth, tenth or eleventh aspect of the disclosure is a method wherein the patient is a pediatric patient. In another embodiment the pediatric patient is a liver transplants patient.

In another embodiment, the method according to any of the above-described embodiments of the ninth, tenth or eleventh aspect of the disclosure is a method wherein the subject at risk of developing a JCV associated disease is immunosuppressed.

In one embodiment the method according to any of the above-described embodiments of the ninth, tenth or eleventh aspect of the disclosure is a method wherein the JCV associated disorder is Progressive Multifocal Leukoencephalopathy (PML).

In an additional embodiment of the ninth, tenth or eleventh aspect of the disclosure, including all the above-described embodiments thereof, the method reduces the likelihood of developing the PML.

In an additional embodiment the method according to the ninth, tenth or eleventh aspect of the disclosure, including all the above-described embodiments thereof, comprises the transplantation of a solid organ from a JCV seropositive donor to a JCV seronegative recipient.

In another embodiment the method according to the ninth, tenth or eleventh aspect of the disclosure, including all the above-described embodiments thereof, is a method wherein the subject at risk of developing a JCV associated disorder will undergo an organ or tissue transplantation.

In one embodiment the method according to the ninth, tenth or eleventh aspect of the disclosure, including all the above-described embodiments thereof, is a method wherein the subject is a JCV-naive seronegative transplant patient who receives an organ from a JCV positive donor.

In an additional embodiment the method according to the ninth, tenth or eleventh aspect of the disclosure, including all the above-described embodiments thereof, is a method wherein the patient is a pediatric patient. In another embodiment the pediatric patient is a liver transplants patient.

In another embodiment, the method according to the ninth, tenth or eleventh aspect of the disclosure, including all the above-described embodiments thereof, is a method wherein the subject is immunosuppressed.

In a twelves aspect of the disclosure the method according to any of the ninth, tenth or eleventh aspect, including all embodiments thereof, is a method wherein the CD40 antagonist is an antibody described herein above.

In another embodiment, the antibody according to the twelves aspect is selected from the group of antibodies described in aspect four above. In one aspect of the invention, the antibody used in the methods according to anyone of the ninth, tenth or eleventh aspect, including all embodiments thereof, is CFZ533.

In a thirteenth aspect of the disclosure, a pharmaceutical composition is provided comprising a therapeutically effective amount of a CD40 antibody, e.g. CFZ533 and one or more pharmaceutically acceptable carriers for use in the methods according to anyone of the ninth, tenth or eleventh aspect, including all embodiments thereof.

In one embodiment of the thirteenth aspect of the disclosure, the route of administration of the pharmaceutical composition comprising the CD40 antibody, e.g. CFZ533, is subcutaneous or intravenous or a combination of subcutaneous or intravenous, as described above in the fifth aspect, including all embodiments thereof.

A fourteens aspect of the disclosure relates to the use of a liquid pharmaceutical composition comprising an anti-CD40 antibody, for the manufacture of a medicament for preventing a JCV associated disorder in a subject at risk of developing such a disorder, wherein the anti-CD40 antibody is an antibody described in the sixth aspect of the invention described above.

In a fifteenth aspect the disclosure relates to an anti-CD40 antibody, e.g. CFZ533, for use according to any of the aspects nine to fourteen, including all embodiment thereof, wherein the anti-CD40 antibody treatment occurs post-transplantation and the antibody is administered so that plasma or serum concentration of the antibody is at least 40 μg/mL.

### BRIEF DESCRIPTION OF THE DRAWINGS

FIG. 1 Representative flow cytometry analysis

Gating strategy for CD3+ T cells and CD19+ B cells by flow cytometry. Cells were stained with CD3 and CD19 antibodies, and analyzed by flow cytometry. First, all acquired cells were plotted in the dot plot on the forward scatter (FCS)/side scatter (SSC) and then further gated either on CD3+ T cells or CD19+ B cells (purple circles). Different conditions are shown from one representative donor ((first row: PBMC+medium-EBV (left plots), PBMC+medium+EBV (right plots); second row: PBMC+0.1 μM CsA+EBV (left plots), PBMC+50 μg/mL Belatacept+EBV (right plots); third row: PBMC+50 μg/mL hIgG1+EBV (left plots), PBMC+50 μg/mL CFZ533+EBV (right plots)).

FIG. 2 CD3+ T cell counts from four different donors: CD3⁺ T cell counts from the PBMC regression assay after 14 days of culture measured by flow cytometry. Antibodies (in μg/mL) and CsA (in μM) were given in combination with EBV supernatants (except the negative control=medium w/o EBV). Dotted line was set to the condition ‘medium+EBV’. Data are shown as average+SEM (n=4).

FIG. 3 CD19⁺ B cell counts from four different donors: CD19⁺ B cell counts from the PBMC regression assay after 14 days of culture measured by flow cytometry. Antibodies (in μg/mL) and CsA (in μM) were given in combination with EBV supernatants (except the negative control=medium w/o EBV). Dotted line was set to the condition ‘medium+EBV’. Data are shown as average+SEM (n=4).

FIG. 4 is a graph showing preliminary simulated pharmacokinetics profiles before study started.

FIG. 5 shows the gating strategy for T cell proliferation by flow cytometry. Cells were stained for CD3, CD4 and CD8 including a viability marker and analyzed by flow cytometry for T cell proliferation. First row from left to right: All acquired cells are shown in the FCS/SSC dot plot and gated on the total viable cells (viability marker) in a histogram plot. From the alive cells, CD3⁺ T cells were chosen and T cell proliferation was identified by gating on Cell Tracer Violet-positive cells. Second row: From the total viable cells, a quadrant was placed to identify CD4⁺ and CD8⁺ T cells, and analyzed for Cell Tracer Violet labelling.

FIG. 6 outlines the in vitro EBV-B cell/T cell co-culture model. The co-culture model consists of two phases: ‘priming’ phase (seven days of culture) and ‘recall’ phase (additional four days of culture). EBV-B cells or primary B cells were used together with autologous T cells, and incubated with either an IgG1 isotype, anti-CD40 (CFZ533) or an anti-CTL-A4 antibody (Belatacept) at four different concentrations (10, 50, 100 and 200 μg/mL or medium). Medium or antibodies were added either at the ‘priming’ phase, ‘recall’ phase or at both phases (‘priming and recall’). After 11 days of co-culture, cells were analyzed by flow cytometry for T cell proliferation and by ELISA for IFNγ production.

FIG. 7 provides CD3+ T cell proliferation data expressed as ratio to corresponding control of co-cultures using EBV-seropositive EBV B-cells and autologous T cells after 11 days of culture measured by flow cytometry. Antibodies (μg/mL) were given at either the ‘priming’ phase (left hand side), ‘recall’ phase (middle part) or ‘priming and recall’ phase (left hand side). Dotted line was set to one (negative control; 0 μg/mL). Data are shown as average+SEM (n=3-10). Numbers above each column represent total numbers of individuals tested.

FIG. 8 provides CD4+ T cell proliferation data expressed as ratio to corresponding control of co-cultures using EBV-seropositive EBV B-cells and autologous T cells after 11 days of culture measured by flow cytometry. Antibodies (μg/mL) were given at either the ‘priming’ phase (left hand side), ‘recall’ phase (middle part) or ‘priming and recall’ phase (left hand side). Dotted line was set to one (negative control; 0 μg/mL). Data are shown as average+SEM (n=3-10). Numbers above each column represent total numbers of individuals tested.

FIG. 9 provides CD8+ T cell proliferation data expressed as ratio to corresponding control of co-cultures using EBV-seropositive EBV B-cells and autologous T cells after 11 days of culture measured by flow cytometry. Antibodies (μg/mL) were given at either the ‘priming’ phase (left hand side), ‘recall’ phase (middle part) or ‘priming and recall’ phase (left hand side). Dotted line was set to one (negative control; 0 μg/mL). Data are shown as average+SEM (n=3-10). Numbers above each column represent total numbers of individuals tested.

FIG. 10 provides CD3+ T cell proliferation data expressed as ratio to corresponding control of co-cultures using EBV-seronegative EBV B-cells and autologous T cells after 11 days of culture measured by flow cytometry. Antibodies (μg/mL) were given at either the ‘priming’ phase (left hand side), ‘recall’ phase (middle part) or ‘priming and recall’ phase (left hand side). Dotted line was set to one (negative control; 0 μg/mL). Data are shown as average+SEM (n=3-7). Numbers above each column represent total numbers of individuals tested.

FIG. 11 provides CD4+ T cell proliferation data in cultures prepared from cells of EBV-seronegative donors. Analyzing CD8+ T cell proliferation (FIG. 12), Belatacept had only a reducing effect with 10 and 50 μg/mL at the ‘recall’ only and ‘priming and recall’ condition whereas 100 and 200 μg/mL were as the vehicle control (0 μg/mL). Interestingly, when belatacept was added only at the ‘priming’ phase, Belatacept increased CD8+ T cell proliferation with a dose of 100 and 200 μg/mL. CFZ533 had no major effects on the CD8+ T cell proliferation except a slight not significant increase at 10 μg/mL (‘priming’ only and ‘priming and recall’ condition) and 50 μg/mL (‘recall’ only condition) as seen for the CD4+ T cell proliferation compared to vehicle control (0 μg/mL).

FIG. 12 provides CD8+ T cell proliferation data in cultures prepared from cells of EBV-seronegative donors. CD8+ T cell proliferation data expressed as ratio to corresponding control of co-cultures using EBV-seronegative EBV B-cells and autologous T cells after 11 days of culture measured by flow cytometry. Antibodies (μg/mL) were given at either the ‘priming’ phase (left hand side), ‘recall’ phase (middle part) or ‘priming and recall’ phase (left hand side). Dotted line was set to one (negative control; 0 μg/mL). Data are shown as average+SEM (n=3-7). Numbers above each column represent total numbers of individuals tested.

FIG. 13 provides IFNγ cytokine levels in cultures prepared from cells of EBV-seropositive donors. IFNγ cytokine levels of EBV-seropositive EBV-B cell/autologous T cell co-culture supernatants after 11 days of incubation expressed as ratio to corresponding control. Antibodies were given either at the ‘priming’ phase (left hand side) or at ‘recall’ phase (middle part) or at both phases (‘priming and recall’; left hand side). Antibodies concentrations was used as μg/mL. Dotted line was set for the negative control (0 μg/mL). Data are shown as average+SEM (n=3-9). Numbers above each column represent total numbers of individual tested.

FIG. 14 provides IFNγ cytokine levels in cultures prepared from cells of EBV-seronegative donors. IFNγ cytokine levels of EBV-seronegative EBV-B cell/autologous T cell co-culture supernatants after 11 days of incubation expressed as ratio to corresponding control. Antibodies were given either at the ‘priming’ phase (left hand side) or at ‘recall’ phase (middle part) or at both phases (‘priming and recall’; left hand side). Antibodies concentrations was used as μg/mL. Dotted line was set for the negative control (0 μg/mL). Data are shown as average+SEM (n=3-6). Numbers above each column represent total numbers of individual tested.

FIG. 15 provides CD3+ T cell counts from the PBMC regression assay after 14 days of culture measured by flow cytometry. Antibodies (in μg/mL) and CsA (in μM) were given in combination with EBV supernatants (except the negative control=medium w/o EBV). Dotted horizontal line was set to the condition ‘medium+EBV’. Data are shown as mean+SEM (n=4).

FIG. 16 provides CD3⁺ T cell counts from the NK cell-depleted PBMC regression assay after 14 days of culture measured by flow cytometry. Antibodies (in μg/mL) and CsA (in μM) were given in combination with EBV supernatants (except the negative control=medium w/o EBV). Dotted horizontal line was set to the condition ‘medium+EBV’. Data are shown as mean+SEM (n=4).

FIG. 17 provides CD19⁺ B cell counts from the PBMC regression assay after 14 days of culture measured by flow cytometry. Antibodies (in μg/mL) and CsA (in μM) were given in combination with EBV supernatants (except the negative control=medium w/o EBV). Dotted horizontal line was set to the condition ‘medium+EBV’. Data are shown as mean+SEM (n=4); and CD19⁺ B cell counts from the NK cell-depleted PBMC regression assay after 14 days of culture measured by flow cytometry. Antibodies (in μg/mL) and CsA (in μM) were given in combination with EBV supernatants (except the negative control=medium w/o EBV). Dotted horizontal line was set to the condition ‘medium+EBV’. Data are shown as mean+SEM (n=4).

### GENERAL DEFINITIONS

As used herein, CD40 refers to cluster of differentiation 40, also called tumor necrosis factor receptor superfamily member 5. The term CD40 refers to human CD40, for example as defined in SEQ ID NO: 19, unless otherwise described.

The term “about” in relation to a numerical value x means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

The term “comprising” encompasses “including” as well as “consisting,” e.g., a composition “comprising” X may consist exclusively of X or may include something additional, e.g., X+Y.

The term “antibody” or “anti-CD40 antibody” and the like as used herein refers to whole antibodies that interact with (e.g., by binding, steric hindrance, stabilizing/destabilizing, spatial distribution) a CD40. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FRs). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelid antibodies, or chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass, preferably IgG and most preferably IaG1. Exemplary antibodies include CFZ533 (herein also designated mAb1) and mAb2, as set forth in Table 1.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively. In particular, the term “antibody” specifically includes an IgG-scFv format.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). The term “monoclonal antibody” or “monoclonal antibody composition” as used herein refer to a preparation of antibody molecules of single molecular composition. The term “human antibody”, as used herein, is intended to include antibodies having variable regions in which both the framework and CDR regions are derived from sequences of human origin. A “human antibody” need not be produced by a human, human tissue or human cell. The human antibodies of the disclosure may include amino acid residues not encoded by human sequences (e.g., mutations introduced by random or site-specific mutagenesis in vitro, by N-nucleotide addition at junctions in vivo during recombination of antibody genes, or by somatic mutation in vivo).

The term “Fc region” as used herein refers to a polypeptide comprising the CH3, CH2 and at least a portion of the hinge region of a constant domain of an antibody. Optionally, an Fc region may include a CH4 domain, present in some antibody classes. An Fc region, may comprise the entire hinge region of a constant domain of an antibody. In one embodiment, the binding molecule used in the method of the invention comprises an Fc region and a CH1 region of an antibody. In one embodiment, the binding molecule used in the method of the invention comprises an Fc region CH3 region of an antibody. In another embodiment, the binding molecule used in the method of the invention comprises an Fc region, a CH1 region and a C_{kappa/lambda} region from the constant domain of an antibody. In one embodiment, a binding molecule used in the method of the invention comprises a constant region, e.g., a heavy chain constant region.

As used herein, the term “ADCC” or “antibody-dependent cellular cytotoxicity” activity refers to cell depleting activity. ADCC activity can be measured by the ADCC assay as well known to a person skilled in the art.

As used herein, the term “silent” antibody refers to an antibody that exhibits no or low ADCC activity as measured in an ADCC assay.

In one embodiment, the term “no or low ADCC activity” means that the silent antibody exhibits an ADCC activity that is below 50% specific cell lysis, for example below 10% specific cell lysis as measured in a standard ADCC assay. No ADCC activity means that the silent antibody exhibits an ADCC activity (specific cell lysis) that is below 1%.

Silenced effector functions can be obtained by mutation in the Fc region of the antibodies and have been described in the art: LALA and N297A (Strohl, W., 2009, Curr. Opin. Biotechnol. vol. 20(6):685-691); and D265A (Baudino et al., 2008, J. Immunol. 181:6664-69; Strohl, W., supra). Examples of silent Fc IgG1 antibodies comprise the so-called LALA mutant comprising L234A and L235A mutation in the IgG1 Fc amino acid sequence. Another example of a silent IgG1 antibody comprises the D265A mutation. Another silent IgG1 antibody comprises the N297A mutation, which results in aglycosylated/non-glycosylated antibodies.

The term “treatment” or “treat” is herein defined as the application or administration of e.g. an anti-CD40 antibody or protein according to the invention, for example, mAb1 or mAb2 antibody, to a subject, or application or administration a pharmaceutical composition comprising said anti-CD40 antibody to an isolated tissue or cell line from a subject, where the purpose is controlling EBV infection or preventing EBV infection.

By “treatment” is also intended the application or administration of a pharmaceutical composition comprising for example mAb1 or mAb2 antibody, to a subject, or application or administration of a pharmaceutical composition comprising said anti-CD40 antibody to an isolated tissue or cell line from a subject, where the subject is at risk of developing an EBV-associated disease.

As used herein, a subject is “in need of” a treatment if such subject would benefit biologically, medically or in quality of life from such treatment. The term “subject” as used herein can be a mammal, e.g., a primate, preferably a higher primate, e.g., a human (e.g., a patient having, or at risk of having, a disorder described herein, like transplant patients). As used herein, the term “administration” or “administering” of the subject compound means providing a CD40 antagonist, e.g. CFZ533 to a subject in need of treatment. Administration “in combination with” one or more further therapeutic agents includes simultaneous (concurrent) and consecutive administration in any order, and in any route of administration.

As used herein, a “therapeutically effective amount” refers to an amount of CD40 antagonist, e.g. an anti-CD40 antibody or antigen binding fragment thereof, for example mAb1, that is effective, upon single or multiple dose administration to a patient (such as a human) for the purpose is controlling EBV infection or preventing EBV infection.

When applied to an individual active ingredient (e.g., an anti-CD40 antibody, e.g., mAb1) administered alone, the term refers to that ingredient alone. When applied to a combination, the term refers to combined amounts of the active ingredients that result in the therapeutic effect, whether administered in combination, serially or simultaneously.

The phrase “therapeutic regimen” means the regimen used to treat an illness or to prevent a disease condition or the development of a disease, e.g., the dosing used during the prevention of an EBV infection, to reduce the EBV load or the development of an EBV-associated disease. A therapeutic regimen may include an induction regimen, a loading regimen and a maintenance regimen.

The phrase “loading regimen” or “loading period” refers to a treatment regimen (or the portion of a treatment regimen) that is used for the initial treatment of a disease. In some embodiments, the disclosed methods, uses, kits, processes and regimens (e.g., methods of preventing graft loss in solid organ transplantation) employ a loading regimen. In some cases, the loading period is the period until maximum efficacy is reached. The general goal of a loading regimen is to provide a high level of drug to a patient during the initial period of a treatment regimen. An induction regimen may employ (in part or in whole) a “loading regimen” or “loading dosing”, which may include administering a greater dose of the drug than a physician would employ during maintenance regimen, administering a drug more frequently than a physician would administer the drug during a maintenance regimen, or both. Dose escalation may occur during or after an induction regimen.

The phrase “maintenance regimen” or “maintenance period” refers to a treatment regimen (or the portion of a treatment regimen) that is used for the maintenance of a patient during treatment of an illness, e.g., to keep the patient in remission for long periods of time (months or years) following the induction period. In some embodiments, the disclosed methods, uses and regimens employ a maintenance regimen. A maintenance regimen may employ continuous therapy (e.g., administering a drug at a regular intervals, e.g., weekly, monthly [every 4 weeks], yearly, etc.) or intermittent therapy (e.g., interrupted treatment, intermittent treatment, treatment at relapse, or treatment upon achievement of a particular predetermined criteria [e.g., pain, disease manifestation, etc.]). Dose escalation may occur during a maintenance regimen.

The phrase “means for administering” is used to indicate any available implement for systemically administering a drug to a patient, including, but not limited to, a pre-filled syringe, a vial and syringe, an injection pen, an autoinjector, an i.v. drip and bag, a pump, a patch pump, etc. With such items, a patient may self-administer the drug (i.e., administer the drug on their own behalf) or a physician may administer the drug.

### DETAILED DESCRIPTION OF THE INVENTION

Epstein-Barr virus (EBV) is a human herpesvirus 4 (HHV4) and belongs to the genus Lymphocryptovirus within the subfamily of gamma herpes viruses. These viruses establish latent infections of their host cells and induce proliferation of the latently infected cells (reviewed in Roizman B. Herpesviridae: general description, taxonomy and classification. In: Roizman B, editor. The herpesviruses. London: Plenum Press, 1996:1_/23). EBV is associated with a still growing spectrum of clinical disorders, ranging from acute and chronic inflammatory diseases to lymphoid and epithelial malignancies. Epstein-Barr virus is associated with lymphoproliferative diseases, a type of diseases in which different types of lymphoid cells like T-cells, B-cells or natural killer (NK) cells are infected with the Epstein-Barr virus. The infected cells divide excessively and develop various lymphoproliferative disorders (LPD, non-cancerous, pre-cancerous, and cancerous). These LPDs include infectious mononucleosis and subsequent disorders that may occur thereafter. Non-LPD but EBV-associated diseases include malignancies, sarcomas, multiple sclerosis, systemic lupus erythematosus, Hodgkin and non-Hodgkin lymphomas, nasopharyngeal carcinoma, gastric carcinoma, leiomyosarcoma and the “Alice in Wonderland syndrome” (Middeldorp et al., Critical Reviews in Oncology/Hematology 45 (2003) 1-/36 2003).

This invention is based on the surprising finding that a CD40 antagonist does not impair EBV control in vitro, in contrast to CsA and Belatacept. CD40 antagonists are currently in clinical development for inhibition of transplant organ rejection and therapy for autoimmune diseases. Hence, the surprising finding that an immunosuppressive CD40 antagonist does not impair EBV control is the precondition and basis for the development of therapeutic methods, like methods for organ transplantation in patients being at risk of developing EBV-associated diseases like post-transplant iymphoproliferative disease (PTLD). Therefore, the disclosure relates to a method of preventing an EBV-associated disorder as well as to a method of controlling EBV load in a subject, in particular in a subject being at risk of developing such a disorder, comprising administering to said subject a therapeutically effective dose of a CD40 antagonist.

The term “EBV control” or “controlling EBV infection” as used herein refers to the outcome of the treatment of a subject, in particular treatment of a patient, more particular a patient in need of immuno-suppression, even more particular a patient that receives an organ or tissue transplantation, with a therapeutically effective dose of a CD40 antagonist as disclosed herein (e.g. CFZ533), wherein EBV control is achieved if anyone of the following criteria is fulfilled (i) an EBV latency 0 status in the patient, (Ruf et al., Transplantation & Volume 97, Number 9, May 15, 2014), (ii) an EBV latency I status or (iii) no serologic evidence of an active EBV infection (e.g. no detection of EBV antibodies or expressed EBV proteins using specific antibodies). In a preferred embodiment the above listed EBV control criteria (i), (ii) and/or (iii)/latency status is maintained for at least a period of 6 month, or for at least a period of 9 month, or for at least a period of 12 month, or for at least a period of 15 month, or for at least a period of 18 month, or for at least a period of 21 month, or for at least a period of 24 month, or for at least a period of 3 years, or for at least a period of 4 years, or for at least a period of 5 years, or for at least a period of 6 years, or for at least a period of 7 years, or for at least a period of 8 years or longer after the transplantation has taken place.

The term EBV control or controlling EBV infection also refers to an outcome of the above described treatment, wherein the EBV load in whole blood of a treated subject is below 5000 copies of the EBV genome/μg DNA, or below 4500 copies/μg DNA, or below 4000 copies/μg DNA, or below 3500 copies/μg DNA, or below 3000 copies/μg DNA, or below 2500 copies/μg DNA, or below 2000 copies/μg DNA, or below 1500 copies/μg DNA, or below 1000 copies/μg DNA. In a preferred embodiment the above described EBV load in whole blood is maintained for at least a period of 6 month, or for at least a period of 9 month, or for at least a period of 12 month, or for at least a period of 15 month, or for at least a period of 18 month, or for at least a period of 21 month, or for at least a period of 24 month, or for at least a period of 3 years, or for at least a period of 4 years, or for at least a period of 5 years, or for at least a period of 6 years, or for at least a period of 7 years, or for at least a period of 8 years or longer after the transplantation has taken place.

The term EBV control or controlling EBV infection also refers to an outcome of the above described treatment, wherein the EBV load in plasma is below 3000 copies/100 μl, or below 2500 copies/100 μl, or below 2000 copies/100 μl, or below 1500 copies/100 μl, or below 1000 copies/100 μl. In a preferred embodiment the above described EBV load in plasma is maintained for at least a period of 6 month, or for at least a period of 9 month, or for at least a period of 12 month, or for at least a period of 15 month, or for at least a period of 18 month, or for at least a period of 21 month, or for at least a period of 24 month, or for at least a period of 3 years, or for at least a period of 4 years, or for at least a period of 5 years, or for at least a period of 6 years, or for at least a period of 7 years, or for at least a period of 8 years or longer after the transplantation has taken place.

Furthermore, the term EBV control or controlling EBV infection as used herein also refers outcome of the treatment of a subject, in particular wherein the subject is a patient, more particular a patient in need of immuno-suppression, even more particular a patient that receives an organ or tissue transplantation, with a therapeutically effective dose of a CD40 antagonist (e.g. CFZ533) as disclosed herein, wherein the outcome is a reduced EBV titer or EBV load or EBV infection status compared to patient in need of immuno-suppression, more particular a patient that received an organ or tissue transplantation, but has not been treated according to the herein disclosed methods, wherein the EBV load (e.g. EBV DNA load) is reduced by at least by 20%, by at least by 30%, by at least by 40%, by at least by 50%, by at least by 60%, by at least by 70%, by at least by 80%, by at least by 90% or more than 90%. In a preferred embodiment, the reduced EBV load is maintained for at least a period of 6 month, or for at least a period of 9 month, or for at least a period of 12 month, or for at least a period of 15 month, or for at least a period of 18 month, or for at least a period of 21 month, or for at least a period of 24 month, or for at least a period of 3 years, or for at least a period of 4 years, or for at least a period of 5 years, or for at least a period of 6 years, or for at least a period of 7 years, or for at least a period of 8 years or longer after the transplantation has taken place.

The term “prevent” or “preventing” generally refer to prophylactic or preventative treatment; it is concerned about delaying the onset of, or preventing the onset of the disease, disorders and/or symptoms associated thereto. The term “preventing an EBV-associated diseases” as used herein refers to the outcome of the treatment of a subject, in particular wherein the subject is a patient, more particular a patient in need of immuno-suppression, even more particular a patient that receives an organ or tissue transplantation, with a therapeutically effective dose of a CD40 antagonist (e.g. CFZ533) as disclosed herein, wherein the patient does not develop an EBV-associated disease, in particular the patient does not develop lymphoproliferative disorders, (LPD, non-cancerous, pre-cancerous, and cancerous, including infectious mononucleosis and subsequent disorders that may occur thereafter, or non-LPD but EBV-associated diseases including malignancies, sarcomas, multiple sclerosis, systemic lupus erythematosus, post-transplant lymphoproliferative disease and the “Alice in Wonderland syndrome”. The term “preventing an EBV-associated diseases” also refers to the outcome of the treatment of a subject as described above, wherein the patient after organ or tissue transplantation does not develop an EBV-associated disease as described herein for at least a period of 12 month, or for at least a period of 18 month, or for at least a period of 24 month, or for at least a period of 3 years, or for at least a period of 4 years, or for at least a period of 5 years or for at least a period of 6 years, or for at least a period of 7 years, or for at least a period of 8 years or longer. The term “preventing an EBV-associated diseases” also refers to a situation in which a patient after organ transplantation does not develop a post-transplant lymphoproliferative disease for at least a period of 12 month, or for at least a period of 18 month, or for at least a period of 24 month, or for at least a period of 3 years, or for at least a period of 4 years, or for at least a period of 5 years or for at least a period of 6 years, or for at least a period of 7 years, or for at least a period of 8 years or longer. The effect of the prevention of an EBV-associated disease can be assessed by standard routine health checks performed by physicians and other skilled persons using state of the art assays and technologies to diagnose and monitor EBV-associated diseases. The skilled person is aware of respective state of the art diagnosis technologies that can be applied for the above-described purpose. EBV viral load measurement has become a routine test for monitoring transplant recipients at high risk of PTLD. PTLD patients nearly always have high levels of EBV DNA in whole blood and in plasma (Gulley M. L., Tans W., CLINICAL MICROBIOLOGY REVIEWS, April 2010, p. 350-366; Wagner, H. J et al., 2001. Patients at risk for development of posttransplant lymphoproliferative disorder: plasma versus peripheral blood mononuclear cells as material for quantification of Epstein-Barr viral load by using real-time quantitative polymerase chain reaction. Transplantation 72:1012-1019). Indeed, high circulating EBV levels serve as a measure of tumor burden that can be monitored during treatment (Green, M. 2001. Management of Epstein-Barr virus-induced post-transplant lymphoproliferative disease in recipients of solid organ transplantation. Am. J. Transplant. 1:103-108; Tsai, D. E. et al., 2008. EBV PCR in the diagnosis and monitoring of posttransplant lymphoproliferative disorder: results of a two-arm prospective trial. Am. J. Transplant. 8:1016-1024). Even before the onset of signs and symptoms, high EBV levels serve as a harbinger of impending PTLD, thus permitting preemptive intervention to avert illness and halt disease progression.

The EBV-titer, -load or -infection status can be analysed by e.g. measuring the EBV DNA load, wherein the EBV DNA quantification can be analysed in whole blood, plasma and/or B-cells (Ruf et. al., Transplantation & Volume 97, Number 9, May 15, 2014). The skilled person is aware of technologies to analyse the EBV load and infection status in patients. EBV DNA load can be assessed by analysing expression of the EBV genes LMP2, LMP1, EBNA2 and/or BZLF1. The expression analysis of LMP2 is preferred, because the strongest correlations were observed between viral load in B cells or whole blood and LMP2 (Ruf et. al., Transplantation & Volume 97, Number 9, May 15, 2014).

In the last two decades, several studies analyzed EBV gene expression in transplant patients (Qu L, Rowe D T. Epstein-Barr virus latent gene expression in uncultured peripheral blood lymphocytes. J Virol 1992; 66: 3715.9; Qu L, Green M, Webber S, et al. Epstein-Barr virus gene expression in the peripheral blood of transplant recipients with persistent circulating virus loads. J Infect Dis 2000; 182: 1013; Gotoh K, Ito Y, Ohta R, et al. Immunologic and virologic analyses in pediatric liver transplant recipients with chronic high Epstein-Barr virus loads. J Infect Dis 2010; 202: 461; Kasztelewicz B, Jankowska I, Pawlowska J, et al. Epstein-Barr virus gene expression and latent membrane protein 1 gene polymorphism in pediatric liver transplant recipients. J Med Virol 2011; 83: 2182). In one embodiment the disclosure relates to a method of preventing post-transplant lymphoproliferative disease in a subject, comprising administering to said subject a therapeutically effective dose of a CD40 antagonist (e.g. a CD40 antibody like CFZ533), wherein the subject has received an organ transplantation, wherein said patient does not develop a post-transplant lymphoproliferative disease for at least a period of 12 month, or for at least a period of 18 month, or for at least a period of 24 month, or for at least a period of 3 years, or for at least a period of 4 years, or for at least a period of 5 years or for at least a period of 6 years, or for at least a period of 7 years, or for at least a period of 8 years or longer.

In another embodiment the disclosure relates to a method of preventing post-transplant lymphoproliferative disease in a subject, comprising administering to said subject a therapeutically effective dose of a CD40 antagonist (e.g. a CD40 antibody like CFZ533), wherein the subject has received a kidney transplantation, liver transplantation, heart transplantation, lung transplantation, pancreas transplantation, intestine transplantation, bone marrow transplantation or allogeneic haematopoietic stem cell tansplantation, wherein said patient does not develop a post-transplant lymphoproliferative disease for at least a period of 12 month, or for at least a period of 18 month, or for at least a period of 24 month, or for at least a period of 3 years, or for at least a period of 4 years, or for at least a period of 5 years or for at least a period of 6 years, or for at least a period of 7 years, or for at least a period of 8 years or longer.

The disclosure furthermore relates to a method of reducing the likelihood that a subject will develop an EBV-associated disorder, comprising administering to the subject a therapeutically effective dose of a CD40 antagonist. The term “reducing the likelihood” as used herein in the context of the development of an EBV-associated disorder refers to the outcome of the treatment of a subject, in particular wherein the subject is a patient, more particular a patient in need of immuno-suppression, even more particular a patient that receives an organ or tissue transplantation, with a therapeutically effective dose of a CD40 antagonist (e.g. an CD40 antibody like CFZ533) as disclosed herein, wherein the patient has a reduced risk of developing an EBV-associated disorder, wherein the reduced risk is characterized by a reduced EBV titer or EBV load or EBV infection status compared to patient in need of immuno-suppression, more particular a patient that received an organ or tissue transplantation, but has not been treated according to the herein disclosed methods, wherein the EBV titer or EBV load or EBV infection status (e.g. based on EBV titer or EBV load or EBV infection status EBV DNA load) is reduced by at least by 20%, by at least by 30%, by at least by 40%, by at least by 50%, by at least by 60%, by at least by 70%, by at least by 80%, by at least by 90% or more than 90%. In a preferred embodiment, the above described reduced EBV load is maintained for a at least a period of 6 month, or for at least a period of 9 month, or for at least a period of 12 month, or for at least a period of 15 month, or for at least a period of 18 month, or for at least a period of 21 month, or for at least a period of 24 month, or for at least a period of 3 years, or for at least a period of 4 years, or for at least a period of 5 years, or for at least a period of 6 years, or for at least a period of 7 years, or for at least a period of 8 years or longer after the transplantation has taken place.

In order to compare patients being treated according to the herein disclosed method with patient not receiving the same treatment to assess the reduced risk of developing an EBV-associated disorder, the skilled person will have no problem to compare the outcome of the treatment according to the herein disclosed method with available clinical statistics on transplant patients developing EBV-associated disorder. Said clinical statistics outline the occurrence of individual EBV-associated diseases, like preventing post-transplant lymphoproliferative disease, in bigger populations. The average occurrence and the timely onset of said diseases can be compared to the outcome of the herein disclosed treatment methods.

The above described methods are particularly relevant to subjects that will undergo an organ or tissue transplant. In a preferred embodiment the organ or tissue transplant is the transplantation of a solid organ. In one embodiment the solid organ is a kidney. In another embodiment the solid organ is a liver. In an additional embodiment the solid organ is a heart, a lung, a pancreas or intestine. Preferred tissue transplantations are composite tissue transplantations.

Kidney transplant patient who are EBV seronegative receiving an organ from an EBV seropositive donor are considered high-risk patients and the Global Outcomes guidelines (Kidney Disease: Improving Global Outcomes (KDIGO) Transplant Work Group. KDIGO clinical practice guideline for the care of kidney transplant recipients. Am J Transplant. 2009; 9(Suppl 3):S1-S155) recommend reducing immunosuppressive medication in EBV-seronegative patients with an increasing EBV viral load. Hence, the herein disclosed methods are particularly relevant for patients being EBV-naive and wherein the solid organ donor is EBV seropositive. The terms EBV negative, EBV naive and EBV seronegative are used herein interchangeable.

Immunosuppression or immunosuppressed refers to a deliberately induced reduction of the immune system. Immunosuppression is performed to reduce organ rejection reactions in transplant patients. Immunosuppressive agents are widely used in the treatment of immune-mediated diseases and transplantation. The skilled person is well aware of available methods and technologies (Wiseman A., Clin J Am Soc Nephrol 11: 332-343. February, 2016). An unintended consequence is failure to suppress EBV infection in transplant patients leading to the development of EBV-associated diseases. Thus, the herein disclosed methods are particularly relevant for patients receiving immunosuppressive agents, which are well known to the person skilled in the art.

The incidence of PTLD is 1% in adults with kidney transplant and is higher in pediatric patients, which have an observed higher EBV-seronegativity status (49% vs. 8%, respectively (Srivastava T, Zwick D L, Rothberg P G, Warady B A. Posttransplant lymphoproliferative disorder in pediatric renal transplantation. Pediatr Nephrol. 1999; 13:748-54). Thus, the herein disclosed methods are particularly relevant for pediatric patients.

### Anti-CD40 Antibodies

CD40 is a transmembrane glycoprotein constitutively expressed on B cells and antigen-presenting cells (APCs) such as monocytes, macrophages, and dendritic cells (DC). CD40 is also expressed on platelets, and under specific conditions can be expressed on eosinophils and activated parenchymal cells. Ligation of CD40 on B cells results in downstream signaling leading to enhanced B cell survival and important effector functions, including clonal expansion, cytokine secretion, differentiation, germinal center formation, development of memory B cells, affinity maturation, immunoglobulin (Ig) isotype switching, antibody production and prolongation of antigen presentation. CD154-mediated activation of the antigen-presenting cell (APC) also leads to induction of cytokine secretion and expression of surface activation molecules including CD69, CD54, CD80, and CD86 that are involved in the regulation of CD4+T helper cell and CD8+ T cell cross-priming and activation.

CD154 exists in two forms; membrane-bound and soluble. Membrane-bound CD154 is a transmembrane glycoprotein expressed on activated CD4+, CD8+, and T-lymphocytes, mast cells, monocytes, basophils, eosinophils, natural killer (NK) cells, activated platelets and has been reported on B cells. It may also be expressed at low levels on vascular endothelial cells and up-regulated during local inflammation. Soluble CD154 (sCD154) is formed after proteolysis of membrane-bound CD154 and is shed from lymphocytes and platelets following cell activation. Once shed, sCD154 remains functional and retains its ability to bind to the CD40 receptor.

The critical role of CD40/CD154 interactions in vivo are best illustrated by patients suffering from Hyper-Immunoglobulin M (HIGM) as a result of loss of function mutations in CD40 or its ligand. Patients with HIGM present with a severe impairment of T cell dependent antibody responses, lack of B cell memory, and little to no circulating IgG, IgA or IgE. In patients with mutations in CD40 signaling, a similar phenotype and disease presentation has been described (van Kooten and Banchereau 2000).

In one embodiment of the disclosure the CD40 antagonist used in the herein disclosed methods (described in the different aspect, including all embodiments thereof, disclosed herein) is a CD40 antibody. In a particularly preferred embodiment the CD40 antibody is an antibody with silenced ADCC activity.

Anti-CD40 mAbs with silenced ADCC activity have been disclosed in U.S. Pat. Nos. 8,828,396 and 9,221,913, incorporated by reference here in their entirety. Anti-CD40 mAbs with silenced ADCC activity are predicted to have an improved safety profile relative to other anti-CD40 antibodies. CD40 antibodies are known to be suitable for the prevention of graft rejection in solid organ transplantation, and particularly prevention of graft rejection in kidney transplantation or liver transplantation. The anti-CD40 antibodies disclosed herein are suitable for prevention of graft rejection in solid organ transplantation, and particularly prevention of graft rejection in kidney transplantation, liver transplantation, heart transplantation, lung transplantation, pancreas transplantation, intestine transplantation or composite tissue transplantation and at the same time are suitable to be used in the methods disclosed herein.

According to a non-binding hypothesis of the inventors, the two mAbs from U.S. Pat. Nos. 8,828,396 and 9,221,913, designated mAb1 and mAb2, are suitable to be used in the herein disclosed methods of preventing an EBV-associated disorder or methods of controlling EBV load. The antibody mAb1, also called CFZ533, is particularly preferred.

To enable a person skilled in the art to practice the invention, the amino acid and nucleotide sequences of mAb1 and mAb2 are provided in Table 1 below.

Another anti-CD40 mAb known in the art that can be used in accordance with the herein described method is ASKP1240 from Astellas Pharma/Kyowa Hakko Kirin Co, as described e.g. in U.S. Pat. No. 8,568,725B2, incorporated by reference herein.

Yet another anti-CD40 mAb known in the art is BI655064 from Boehringer Ingelheim, as described e.g. in U.S. Pat. No. 8,591,900, incorporated by reference herein.

A further anti-CD40 mAb known in the art is FFP104 by Fast Forward Pharmaceuticals, as described e.g. in U.S. Pat. No. 8,669,352, incorporated by reference herein.

Antibodies with the same mode of action as the above mentioned antibodies, so called biosimilars, are also covered by the disclosure, as will be appreciated by a person skilled in the art.

TABLE 1
Sequence table

| SEQ | | |
|---|---|---|
| ID | Description of | |
| NO: | sequence | Detailed amino acid or nucleotide sequences |
| 1 | HCDR1 of mAb 1 | SYGMH |
| | and mAb2 (Kabat) | |
| 2 | HCDR2 of mAb 1 | VISYEESNRYHADSVKG |
| | and mAb2 (Kabat) | |
| 3 | HCDR3 of mAb 1 | DGGIAAPGPDY |
| | and mAb2 (Kabat) | |
| 4 | LCDR1 of mAb 1 | RSSQSLLYSNGYNYLD |
| | and mAb2 (Kabat) | |
| 5 | LCDR2 of mAb 1 | LGSNRAS |
| | and mAb2 (Kabat) | |
| 6 | LCDR3 of mAb 1 | MQARQTPFT |
| | and mAb2 (Kabat) | |
| 7 | Variable Heavy | QVQLVESGGGVVQPGRSLRLSCAASGFTFSSYGMHWVRQA |
| | chain of mAb1 and | PGKGLEWVAVISYEESNRYHADSVKGRFTISRDNSKITLYLQ |
| | mAb2 | MNSLRTEDTAVYYCARDGGIAAPGPDYWGQGTLVTVSS |
| 8 | Variable light chain | DIVMTQSPLSLTVTPGEPASISCRSSQSLLYSNGYNYLDWYL |
| | of mAb1 and mAb2 | QKPGQSPQVLISLGSNRASGVPDRFSGSGSGTDFTLKISRVEA |
| | | EDVGVYYCMQARQTPFTFGPGTKVDIR |
| 9 | Full length heavy | QVQLVESGGGVVQPGRSLRLSCAASGFTFSSYGMHWVRQA |
| | chain of mAb1 | PGKGLEWVAVISYEESNRYHADSVKGRFTISRDNSKITLYLQ |
| | | MNSLRTEDTAVYYCARDGGIAAPGPDYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKRVEPKSCDKTHTCPPCPAPELLGGPSVFLFPP |
| | | KPKDTLMISRTPEVTCVVVDVSHEDPEVKFNWYVDGVEVH |
| | | NAKTKPREEQYASTYRVVSVLTVLHQDWLNGKEYKCKVSN |
| | | KALPAPIEKTISKAKGQPREPQVYTLPPSREEMTKNQVSLTC |
| | | LVKGFYPSDIAVEWESNGQPENNYKTTPPVLDSDGSFFLYSK |
| | | LTVDKSRWQQGNVFSCSVMHEALHNHYTQKSLSLSPGK |
| 10 | Full length light | DIVMTQSPLSLTVTPGEPASISCRSSQSLLYSNGYNYLDWYL |
| | chain of mAb1 | QKPGQSPQVLISLGSNRASGVPDRFSGSGSGTDFTLKISRVEA |
| | | EDVGVYYCMQARQTPFTFGPGTKVDIRRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC |
| 11 | Full length heavy | QVQLVESGGGVVQPGRSLRLSCAASGFTFSSYGMHWVRQA |
| | chain of mAb2 | PGKGLEWVAVISYEESNRYHADSVKGRFTISRDNSKITLYLQ |
| | | MNSLRTEDTAVYYCARDGGIAAPGPDYWGQGTLVTVSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKRVEPKSCDKTHTCPPCPAPELLGGPSVFLFPP |
| | | KPKDTLMISRTPEVTCVVVAVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSREEMTKNQVSLTC |
| | | LVKGFYPSDIAVEWESNGQPENNYKTTPPVLDSDGSFFLYSK |
| | | LTVDKSRWQQGNVFSCSVMHEALHNHYTQKSLSLSPGK |
| 12 | Full length light | DIVMTQSPLSLTVTPGEPASISCRSSQSLLYSNGYNYLDWYL |
| | chain of mAb2 | QKPGQSPQVLISLGSNRASGVPDRFSGSGSGTDFTLKISRVEA |
| | | EDVGVYYCMQARQTPFTFGPGTKVDIRRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC |
| 13 | Fc region of mAb1 | APELLGGPSVFLFPPKPKDTLMISRTPEVTCVVVDVSHEDPE |
| | | VKFNWYVDGVEVHNAKTKPREEQYASTYRVVSVLTVLHQ |
| | | DWLNGKEYKCKVSNKALPAPIEKTISKAKGQPREPQVYTLP |
| | | PSREEMTKNQVSLTCLVKGFYPSDIAVEWESNGQPENNYKT |
| | | TPPVLDSDGSFFLYSKLTVDKSRWQQGNVFSCSVMHEALHN |
| | | HYTQKSLSLSPGK |
| 14 | Fc region of mAb2 | APELLGGPSVFLFPPKPKDTLMISRTPEVTCVVVAVSHEDPE |
| | | VKFNWYVDGVEVHNAKTKPREEQYNSTYRVVSVLTVLHQ |
| | | DWLNGKEYKCKVSNKALPAPIEKTISKAKGQPREPQVYTLP |
| | | PSREEMTKNQVSLTCLVKGFYPSDIAVEWESNGQPENNYKT |
| | | TPPVLDSDGSFFLYSKLTVDKSRWQQGNVFSCSVMHEALHN |
| | | HYTQKSLSLSPGK |
| 15 | DNA encoding Full | CAGGTGCAGCTGGTGGAATCTGGCGGCGGAGTGGTGCAG |
| | length heavy chain | CCTGGCCGGTCCCTGAGACTGTCTTGCGCCGCCTCCGGCT |
| | of mAb1 | TCACCTTCTCCAGCTACGGCATGCACTGGGTGCGACAGGC |
| | | CCCTGGCAAGGGACTGGAATGGGTGGCCGTGATCTCCTAC |
| | | GAGGAATCCAACAGATACCACGCTGACTCCGTGAAGGGC |
| | | CGGTTCACAATCTCCCGGGACAACTCCAAGATCACCCTGT |
| | | ACCTGCAGATGAACTCCCTGCGGACCGAGGACACCGCCG |
| | | TGTACTACTGCGCCAGGGACGGAGGAATCGCCGCTCCTG |
| | | GACCTGATTATTGGGGCCAGGGCACCCTGGTGACAGTGTC |
| | | CTCCGCTAGCACCAAGGGCCCCTCCGTGTTCCCTCTGGCC |
| | | CCCTCCAGCAAGTCCACCTCTGGCGGCACCGCCGCTCTGG |
| | | GCTGCCTGGTGAAAGACTACTTCCCCGAGCCCGTGACCGT |
| | | GTCCTGGAACTCTGGCGCCCTGACCTCCGGCGTGCACACC |
| | | TTTCCAGCCGTGCTGCAGTCCTCCGGCCTGTACTCCCTGTC |
| | | CTCCGTGGTGACCGTGCCCTCTAGCTCTCTGGGCACCCAG |
| | | ACCTACATCTGCAACGTGAACCACAAGCCCTCCAACACCA |
| | | AGGTGGACAAGCGGGTGGAACCCAAGTCCTGCGACAAGA |
| | | CCCACACCTGTCCCCCCTGCCCTGCCCCTGAACTGCTGGG |
| | | CGGACCTTCCGTGTTCCTGTTCCCCCCAAAGCCCAAGGAC |
| | | ACCCTGATGATCTCCCGGACCCCCGAAGTGACCTGCGTGG |
| | | TGGTGGACGTGTCCCACGAGGACCCTGAAGTGAAGTTCA |
| | | ATTGGTACGTGGACGGCGTGGAAGTGCACAACGCCAAGA |
| | | CCAAGCCCAGAGAGGAACAGTACGCCTCCACCTACCGGG |
| | | TGGTGTCTGTGCTGACCGTGCTGCACCAGGACTGGCTGAA |
| | | CGGCAAAGAGTACAAGTGCAAGGTCTCCAACAAGGCCCT |
| | | GCCTGCCCCCATCGAAAAGACCATCTCCAAGGCCAAGGG |
| | | CCAGCCCCGCGAGCCACAGGTGTACACACTGCCCCCCAG |
| | | CCGGGAAGAGATGACCAAGAACCAGGTGTCCCTGACCTG |
| | | TCTGGTCAAAGGCTTCTACCCCTCCGATATCGCCGTGGAG |
| | | TGGGAGTCCAACGGACAGCCCGAGAACAACTACAAGACC |
| | | ACCCCCCCTGTGCTGGACTCCGACGGCTCATTCTTCCTGT |
| | | ACTCCAAGCTGACCGTGGACAAGTCCCGGTGGCAGCAGG |
| | | GCAACGTGTTCTCCTGCTCCGTGATGCACGAGGCCCTGCA |
| | | CAACCACTACACCCAGAAGTCCCTGTCCCTGAGCCCCGGC |
| | | AAG |
| 16 | DNA encoding Full | GACATCGTGATGACCCAGTCCCCCCTGTCCCTGACCGTGA |
| | length light chain of | CACCTGGCGAGCCTGCCTCTATCTCCTGCAGATCCTCCCA |
| | mAb1 | GTCCCTGCTGTACTCCAACGGCTACAACTACCTGGACTGG |
| | | TATCTGCAGAAGCCCGGCCAGTCCCCACAGGTGCTGATCT |
| | | CCCTGGGCTCCAACAGAGCCTCTGGCGTGCCCGACCGGTT |
| | | CTCCGGCTCTGGCTCTGGCACCGACTTCACACTGAAGATC |
| | | TCACGGGTGGAAGCCGAGGACGTGGGCGTGTACTACTGC |
| | | ATGCAGGCCCGGCAGACCCCCTTCACCTTCGGCCCTGGCA |
| | | CCAAGGTGGACATCCGGCGTACGGTGGCCGCTCCCAGCG |
| | | TGTTCATCTTCCCCCCCAGCGACGAGCAGCTGAAGAGCGG |
| | | CACCGCCAGCGTGGTGTGCCTGCTGAACAACTTCTACCCC |
| | | CGGGAGGCCAAGGTGCAGTGGAAGGTGGACAACGCCCTG |
| | | CAGAGCGGCAACAGCCAGGAGAGCGTCACCGAGCAGGAC |
| | | AGCAAGGACTCCACCTACAGCCTGAGCAGCACCCTGACC |
| | | CTGAGCAAGGCCGACTACGAGAAGCATAAGGTGTACGCC |
| | | TGCGAGGTGACCCACCAGGGCCTGTCCAGCCCCGTGACC |
| | | AAGAGCTTCAACAGGGGCGAGTGC |
| 17 | DNA encoding Full | CAGGTGCAGCTGGTGGAATCTGGCGGCGGAGTGGTGCAG |
| | length heavy chain | CCTGGCCGGTCCCTGAGACTGTCTTGCGCCGCCTCCGGCT |
| | of mAb2 | TCACCTTCTCCAGCTACGGCATGCACTGGGTGCGACAGGC |
| | | CCCTGGCAAGGGACTGGAATGGGTGGCCGTGATCTCCTAC |
| | | GAGGAATCCAACAGATACCACGCTGACTCCGTGAAGGGC |
| | | CGGTTCACAATCTCCCGGGACAACTCCAAGATCACCCTGT |
| | | ACCTGCAGATGAACTCCCTGCGGACCGAGGACACCGCCG |
| | | TGTACTACTGCGCCAGGGACGGAGGAATCGCCGCTCCTG |
| | | GACCTGATTATTGGGGCCAGGGCACCCTGGTGACAGTGTC |
| | | CTCCGCTAGCACCAAGGGCCCCTCCGTGTTCCCTCTGGCC |
| | | CCCTCCAGCAAGTCCACCTCTGGCGGCACCGCCGCTCTGG |
| | | GCTGCCTGGTGAAAGACTACTTCCCCGAGCCCGTGACCGT |
| | | GTCCTGGAACTCTGGCGCCCTGACCTCCGGCGTGCACACC |
| | | TTTCCAGCCGTGCTGCAGTCCTCCGGCCTGTACTCCCTGTC |
| | | CTCCGTGGTGACCGTGCCCTCTAGCTCTCTGGGCACCCAG |
| | | ACCTACATCTGCAACGTGAACCACAAGCCCTCCAACACCA |
| | | AGGTGGACAAGCGGGTGGAACCCAAGTCCTGCGACAAGA |
| | | CCCACACCTGTCCCCCCTGCCCTGCCCCTGAACTGCTGGG |
| | | CGGACCTTCCGTGTTCCTGTTCCCCCCAAAGCCCAAGGAC |
| | | ACCCTGATGATCTCCCGGACCCCCGAAGTGACCTGCGTGG |
| | | TGGTGGCCGTGTCCCACGAGGACCCTGAAGTGAAGTTCA |
| | | ATTGGTACGTGGACGGCGTGGAAGTGCACAACGCCAAGA |
| | | CCAAGCCCAGAGAGGAACAGTACAACTCCACCTACCGGG |
| | | TGGTGTCTGTGCTGACCGTGCTGCACCAGGACTGGCTGAA |
| | | CGGCAAAGAGTACAAGTGCAAGGTCTCCAACAAGGCCCT |
| | | GCCTGCCCCCATCGAAAAGACCATCTCCAAGGCCAAGGG |
| | | CCAGCCCCGCGAGCCACAGGTGTACACACTGCCCCCCAG |
| | | CCGGGAAGAGATGACCAAGAACCAGGTGTCCCTGACCTG |
| | | TCTGGTCAAAGGCTTCTACCCCTCCGATATCGCCGTGGAG |
| | | TGGGAGTCCAACGGACAGCCCGAGAACAACTACAAGACC |
| | | ACCCCCCCTGTGCTGGACTCCGACGGCTCATTCTTCCTGT |
| | | ACTCCAAGCTGACCGTGGACAAGTCCCGGTGGCAGCAGG |
| | | GCAACGTGTTCTCCTGCTCCGTGATGCACGAGGCCCTGCA |
| | | CAACCACTACACCCAGAAGTCCCTGTCCCTGAGCCCCGGC |
| | | AAG |
| 18 | DNA encoding Full | GACATCGTGATGACCCAGTCCCCCCTGTCCCTGACCGTGA |
| | length light chain of | CACCTGGCGAGCCTGCCTCTATCTCCTGCAGATCCTCCCA |
| | mAb2 | GTCCCTGCTGTACTCCAACGGCTACAACTACCTGGACTGG |
| | | TATCTGCAGAAGCCCGGCCAGTCCCCACAGGTGCTGATCT |
| | | CCCTGGGCTCCAACAGAGCCTCTGGCGTGCCCGACCGGTT |
| | | CTCCGGCTCTGGCTCTGGCACCGACTTCACACTGAAGATC |
| | | TCACGGGTGGAAGCCGAGGACGTGGGCGTGTACTACTGC |
| | | ATGCAGGCCCGGCAGACCCCCTTCACCTTCGGCCCTGGCA |
| | | CCAAGGTGGACATCCGGCGTACGGTGGCCGCTCCCAGCG |
| | | TGTTCATCTTCCCCCCCAGCGACGAGCAGCTGAAGAGCGG |
| | | CACCGCCAGCGTGGTGTGCCTGCTGAACAACTTCTACCCC |
| | | CGGGAGGCCAAGGTGCAGTGGAAGGTGGACAACGCCCTG |
| | | CAGAGCGGCAACAGCCAGGAGAGCGTCACCGAGCAGGAC |
| | | AGCAAGGACTCCACCTACAGCCTGAGCAGCACCCTGACC |
| | | CTGAGCAAGGCCGACTACGAGAAGCATAAGGTGTACGCC |
| | | TGCGAGGTGACCCACCAGGGCCTGTCCAGCCCCGTGACC |
| | | AAGAGCTTCAACAGGGGCGAGTGC |
| 19 | Amino acid | MVRLPLQCVLWGCLLTAVHPEPPTACREKQYLINSQCCSLC |
| | sequence of human | QPGQKLVSDCTEFTETECLPCGESEFLDTWNRETHCHQHKY |
| | CD40 | CDPNLGLRVQQKGTSETDTICTCEEGWHCTSEACESCVLHR |
| | | SCSPGFGVKQIATGVSDTICEPCPVGFFSNVSSAFEKCHPWTS |
| | | CETKDLVVQQAGTNKTDVVCGPQDRLRALVVIPIIFGILFAIL |
| | | LVLVFIKKVAKKPTNKAPHPKQEPQEINFPDDLPGSNTAAPV |
| | | QETLHGCQPVTQEDGKESRISVQERQ |

In one embodiment, the anti-CD40 antibody provided for use in the disclosed methods, comprises an immunoglobulin VH domain comprising the amino acid sequence of SEQ ID NO: 7 and an immunoglobulin VL domain comprising the amino acid sequence of SEQ ID NO: 8.

In one embodiment, the anti-CD40 antibody provided for use in the disclosed methods, comprises an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6.

In one embodiment, the anti-CD40 antibody provided for use in the disclosed methods, comprises an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13.

In one embodiment, the anti-CD40 antibody provided for use in the disclosed methods, comprises an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14.

In one embodiment, the anti-CD40 antibody described above for use in the disclosed methods, comprises a silent Fc IgG1 region.

In a preferred embodiment, an anti-CD40 antibody designated mAb1 is provided for use in the disclosed methods. Specifically, mAb1 comprises the heavy chain amino acid sequence of SEQ ID NO: 9 and the light chain amino acid sequence of SEQ ID NO: 10; and mAb2 comprises the heavy chain amino acid sequence of SEQ ID NO: 11 and the light chain amino acid sequence of SEQ ID NO: 12.

CFZ533/Iscalimab is a fully human monoclonal Fc-silent, non-depleting anti-CD40 antibody (IgG1/K) that blocks CD40L (CD154)-induced CD40 signaling and is incapable of mediating antibody-dependent cell-mediated cytotoxicity (ADCC) or complement-dependent cytotoxicity (CDC). Currently, CFZ533/Iscalimab is in clinical development for inhibition of transplant organ rejection and therapy for autoimmune diseases.

In an additional embodiment the disclosure provides a method of preventing an EBV-associated disorder or methods of controlling EBV load using mAb1/CFZ533 or mAb2 in combination with CsA, (Neoral®, Novartis).

In another additional embodiment the disclosure provides a method of preventing an EBV-associated disorder or methods of controlling EBV load using mAb1/CFZ533 or mAb2 in combination with tacrolimus (Tac, FK506, Prograf®, Astellas).

In an additional embodiment the disclosure provides a method of preventing an EBV-associated disorder or methods of controlling EBV load using mAb1/CFZ533 or mAb2 in combination with a mTor inhibitor such as everolimus (Zortress®, Certican®, Novartis.

### 1. Expression Systems

For expression of the light and heavy chains, the expression vector(s) encoding the heavy and light chains are transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the antibodies used in the methods of the invention in either prokaryotic or eukaryotic host cells. Expression of antibodies in eukaryotic cells, for example mammalian host cells, yeast or filamentous fungi, is discussed because such eukaryotic cells, and in particular mammalian cells, are more likely than prokaryotic cells to assemble and secrete a properly folded and immunologically active antibody.

Particularly a cloning or expression vector can comprise either at least one of the following coding sequences (a)-(b), operatively linked to suitable promoter sequences: (a) SEQ ID NO: 15 and SEQ ID NO: 16 encoding respectively the full length heavy and light chains of mAb1; or
(b) SEQ ID NO: 17 and SEQ ID NO: 18 encoding respectively the full length heavy and light chains of mAb2.

Mammalian host cells for expressing the recombinant antibodies used in the method of the invention include Chinese Hamster Ovary (CHO cells) (including dhfr-CHO cells, described Urlaub and Chasin, 1980 Proc. Natl. Acad. Sci. USA 77:4216-4220 used with a DH FR selectable marker, e.g., as described in R. J. Kaufman and P. A. Sharp, 1982 Mol. Biol. 159:601-621), CHOK1 dhfr+ cell lines, NSO myeloma cells, COS cells and SP2 cells.

In particular, for use with NSO myeloma cells, another expression system is the GS gene expression system shown in PCT Publications WO 87/04462, WO 89/01036 and EP0338841.

When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods (See for example Abhinav et al. 2007, Journal of Chromatography 848: 28-37).

The host cells may be cultured under suitable conditions for the expression and production of mAb1 or mAb2.

### 2. Pharmaceutical Compositions

Therapeutic antibodies are typically formulated either in aqueous form ready for administration or as lyophilisate for reconstitution with a suitable diluent prior to administration. An anti-CD40 antibody may be formulated either as a lyophilisate, or as an aqueous composition, for example in pre-filled syringes.

Suitable formulation can provide an aqueous pharmaceutical composition or a lyophilisate that can be reconstituted to give a solution with a high concentration of the antibody active ingredient and a low level of antibody aggregation for delivery to a patient. High concentrations of antibody are useful as they reduce the amount of material that must be delivered to a patient. Reduced dosing volumes minimize the time taken to deliver a fixed dose to the patient. The aqueous compositions with high concentration of anti-CD40 antibodies are particularly suitable for subcutaneous administration.

The anti-CD40 antibody may be used as a pharmaceutical composition when combined with a pharmaceutically acceptable carrier. Such a composition may contain, in addition to an anti-CD40 antibody such as mAb1 or mAb2, carriers, various diluents, fillers, salts, buffers, stabilizers, solubilizers, and other materials well known in the art. The characteristics of the carrier will depend on the route of administration. The pharmaceutical compositions for use in the disclosed methods may also contain additional therapeutic agents for treatment of the particular targeted disorder.

In one specific embodiment the composition used in the methods disclosed herein is a lyophilized formulation prepared from an aqueous formulation having a pH of 6.0 and comprising:
(i) 150 mg/mL mAb1 or mAb2
(ii) 270 mM sucrose as a stabilizer,
(iii) 30 mM L-histidine as a buffering agent, and
(iv) 0.06% Polysorbate 20 as a surfactant.

In another specific embodiment the pharmaceutical composition used in the methods disclosed herein is an aqueous pharmaceutical composition has a pH of 6.0 and comprising:
(i) 150 mg/mL mAb1 or mAb2
(ii) 270 mM sucrose as a stabilizer,
(iii) 30 mM L-histidine as a buffering agent, and
(iv) 0.06% Polysorbate 20 as a surfactant.

In another specific embodiment the composition used in the method disclosed herein is a lyophilized or liquid formulation comprising:
(i) mAb1 or mAb2
(ii) sucrose as a stabilizer,
(iii) L-histidine as a buffering agent, and
(iv) Polysorbate 20 as a surfactant and at least one additional active pharmaceutical ingredient selected from the group consisting of a calcineurin inhibitor (CNI) such as cyclosporine (e.g. CsA, Neoral®, Novartis) or tacrolimus (e.g. Tac, FK506, Prograf®, Astellas), a lymphocyte proliferation inhibitor such as mycophenolic acid (e.g. MPA; Myfortic®, Novartis) or mycophenolate mofetil (e.g. MMF; CellCept®, Roche) or proliferation signal inhibitor such as everolimus (e.g. Zortress®, Certican®, Novartis) or sirolimus (e.g. Rapamune®, Pfizer) or a T cell co-stimulation blocker such as belatacept (e.g. Nulojix®, BMS).

### 3. Route of Administration

Typically, the antibodies or proteins are administered by injection, for example, either intravenously, intraperitoneally, or subcutaneously. Methods to accomplish this administration are known to those of ordinary skill in the art. It may also be possible to obtain compositions that may be topically or orally administered, or which may be capable of transmission across mucous membranes. As will be appreciated by a person skilled in the art, any suitable means for administering can be used, as appropriate for a particular selected route of administration.

Examples of possible routes of administration include parenteral, (e.g., intravenous (i.v. or I.V. or iv or IV), intramuscular (IM), intradermal, subcutaneous (s.c. or S.C. or sc or SC), or infusion), oral and pulmonary (e.g., inhalation), nasal, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

The parenteral preparation can be enclosed in ampoules, disposable syringes, or multiple dose vials made of glass or plastic.

An anti-CD40 therapy can optionally be initiated by administering a “loading dose/regimen” of the antibody or protein used in the methods of the invention to the subject in need of anti-CD40 therapy. By “loading dose/regimen” is intended an initial dose/regimen of the anti-CD40 antibody or protein used in the methods of the invention that is administered to the subject, where the dose of the antibody or protein used in the methods of the invention administered falls within the higher dosing range (i.e., from about 10 mg/kg to about 50 mg/kg, such as about 30 mg/kg). The “loading dose/regimen” can be administered as a single administration, for example, a single infusion where the antibody or antigen-binding fragment thereof is administered IV, or as multiple administrations, for example, multiple infusions where the antibody or antigen-binding fragment thereof is administered IV, so long as the complete “loading dose/regimen” is administered within about a 24-hour period (or within the first month if multiple intravenous administration are needed, based on the severity of the disease). Following administration of the “loading dose/regimen”, the subject is then administered one or more additional therapeutically effective doses of the anti-CD40 antibody. Subsequent therapeutically effective doses can be administered, for example, according to a weekly dosing schedule, or once every two weeks (biweekly), once every three weeks, or once every four weeks. In such embodiments, the subsequent therapeutically effective doses generally fall within the lower dosing range (i.e. about 0.003 mg/kg to about 30 mg/kg, such as about 10 mg/kg, e.g 10 mg/kg).

Alternatively, in some embodiments, following the “loading dose/regimen”, the subsequent therapeutically effective doses of the anti-CD40 antibody are administered according to a “maintenance schedule”, wherein the therapeutically effective dose of the CD40 antibody is administered weekly, bi-weekly, or once a month, once every 6 weeks, once every two months, once every 10 weeks, once every three months, once every 14 weeks, once every four months, once every 18 weeks, once every five months, once every 22 weeks, once every six months, once every 7 months, once every 8 months, once every 9 months, once every 10 months, once every 11 months, or once every 12 months. In such embodiments, the therapeutically effective doses of the anti-CD40 antibody fall within the lower dosing range (i.e. about 0.003 mg/kg to about 30 mg/kg, such as about 10 mg/kg, e.g. 10 mg/kg), particularly when the subsequent doses are administered at more frequent intervals, for example, once every two weeks to once every month, or within the higher dosing range (i.e., from 10 mg/kg to 50 mg/kg, such as 30 mg/kg), particularly when the subsequent doses are administered at less frequent intervals, for example, where subsequent doses are administered one month to 12 months apart.

The timing of dosing is generally measured from the day of the first dose of the active compound (e.g., mAb1), which is also known as “baseline”. However, different health care providers use different naming conventions.

Notably, week zero may be referred to as week 1 by some health care providers, while day zero may be referred to as day one by some health care providers. Thus, it is possible that different physicians will designate, e.g., a dose as being given during week 3/on day 21, during week 3/on day 22, during week 4/on day 21, during week 4/on day 22, while referring to the same dosing schedule. For consistency, the first week of dosing will be referred to herein as week 0, while the first day of dosing will be referred to as day 1.

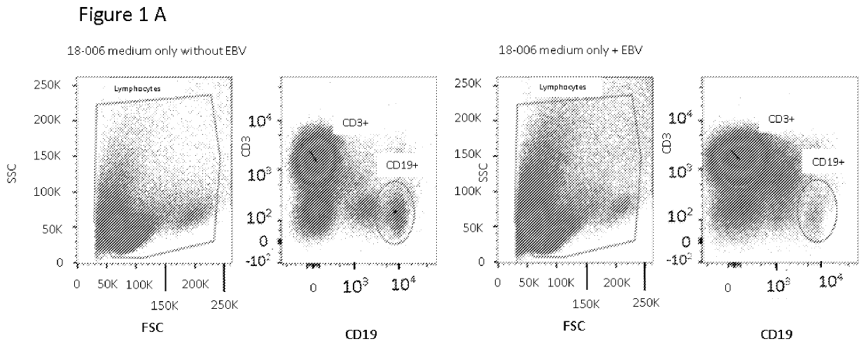

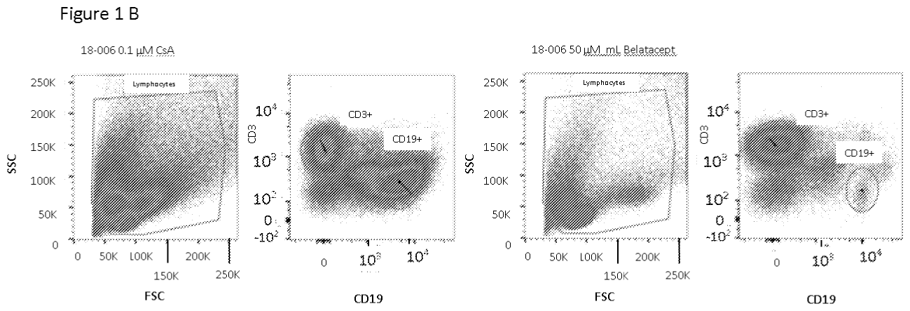

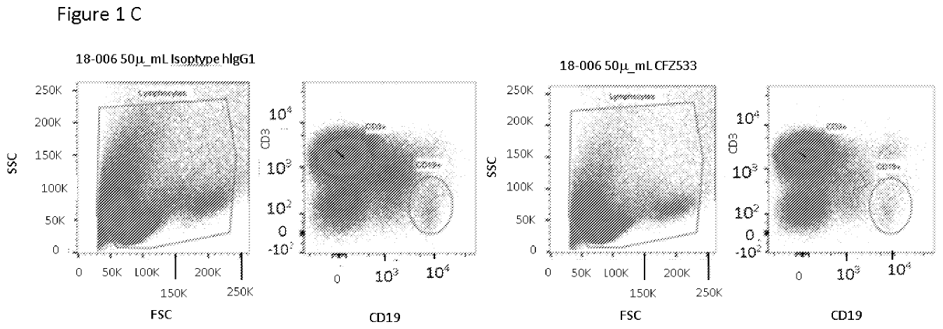

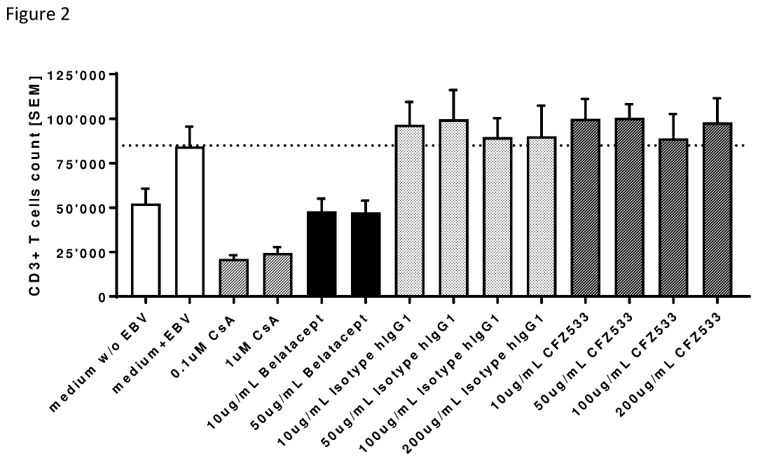

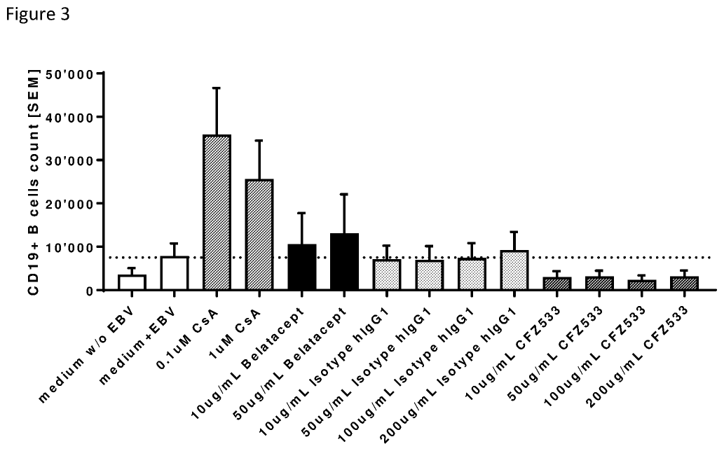

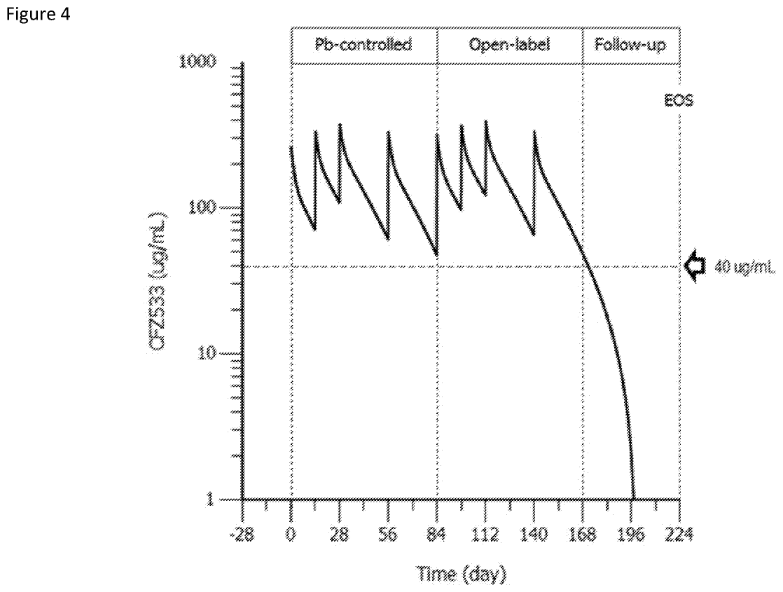

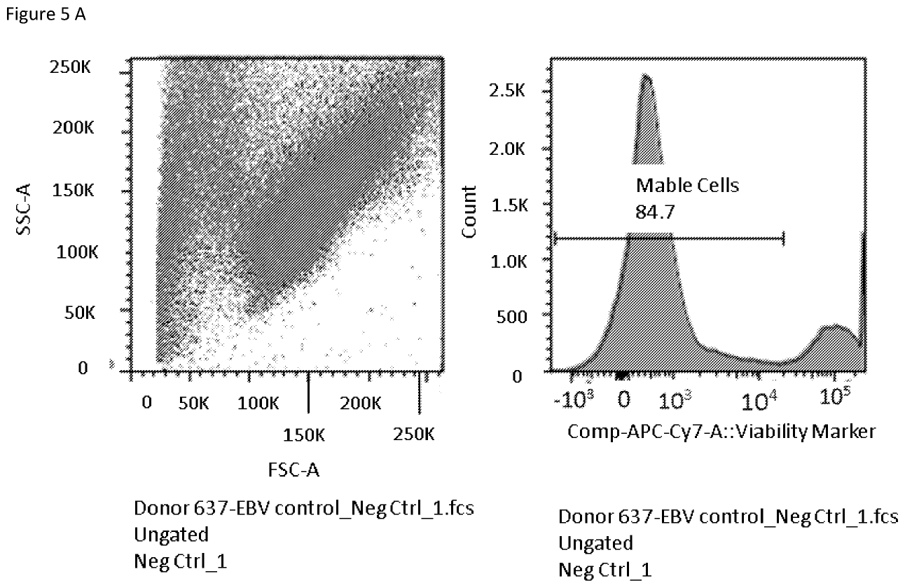

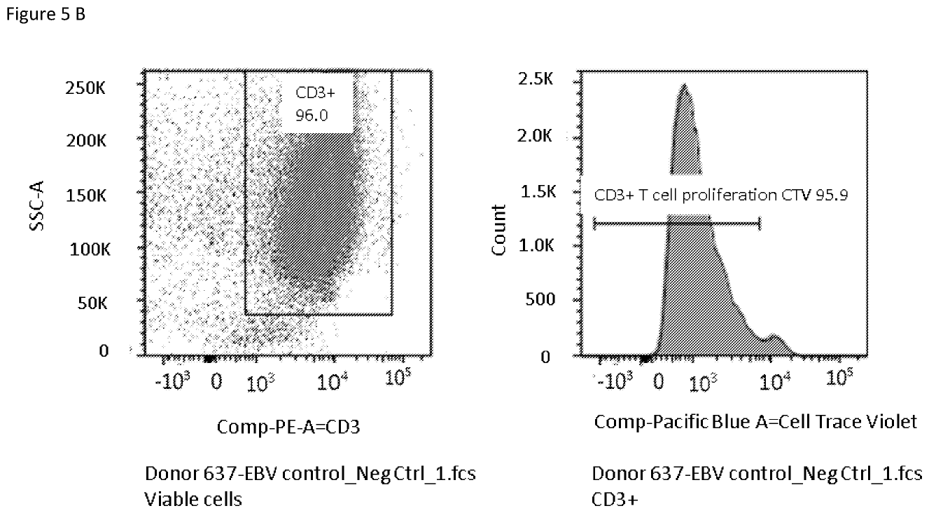

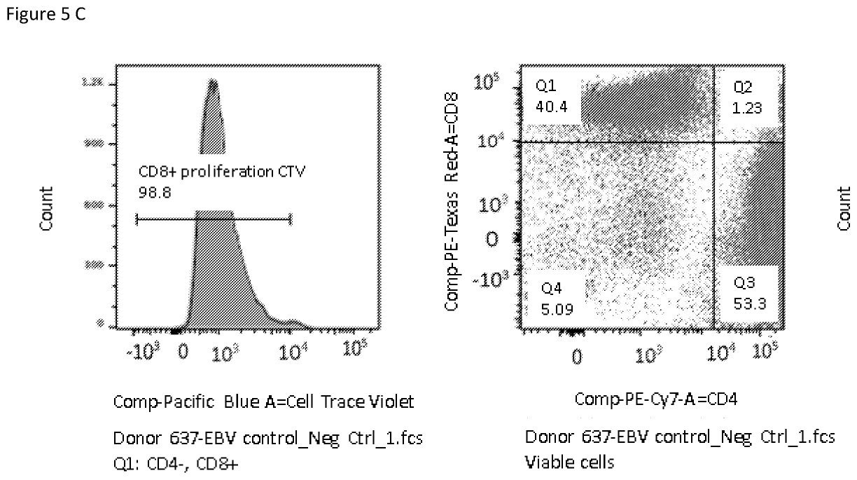

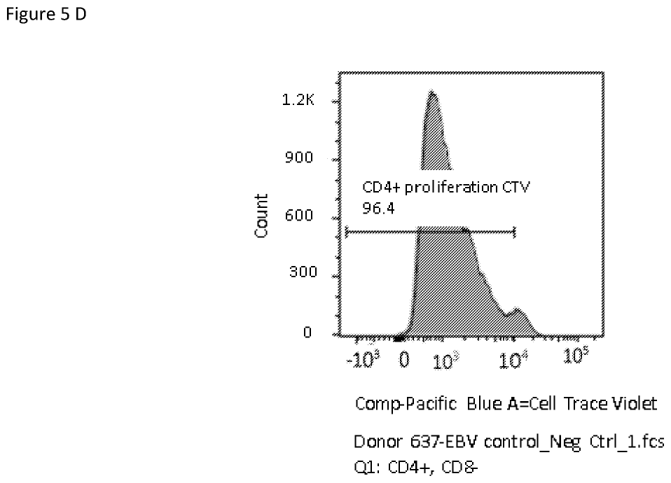

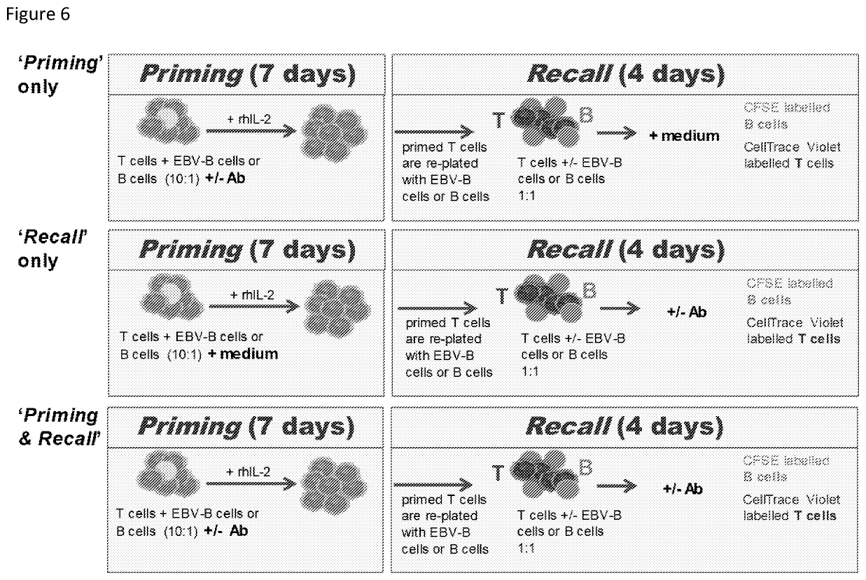

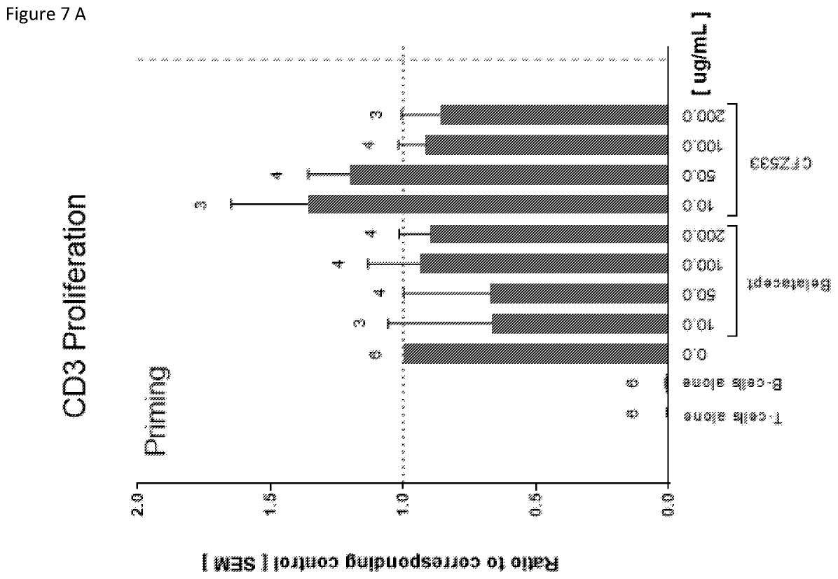

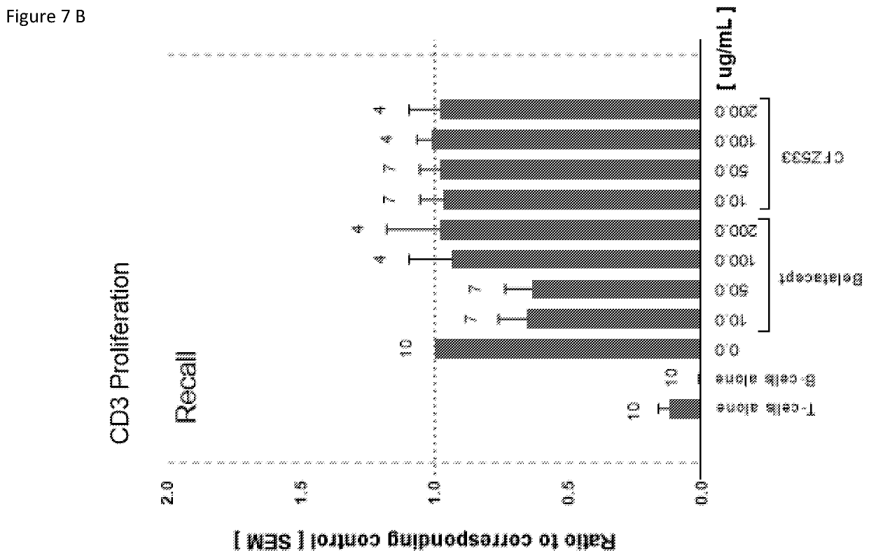

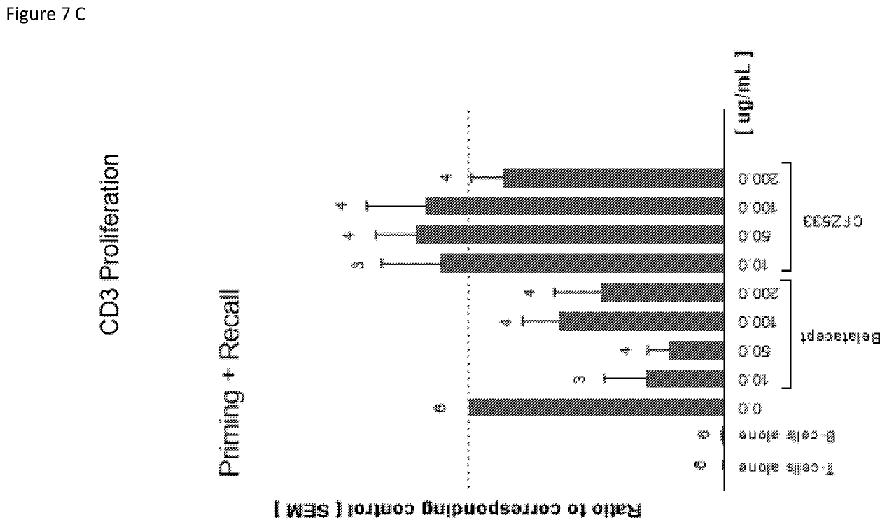

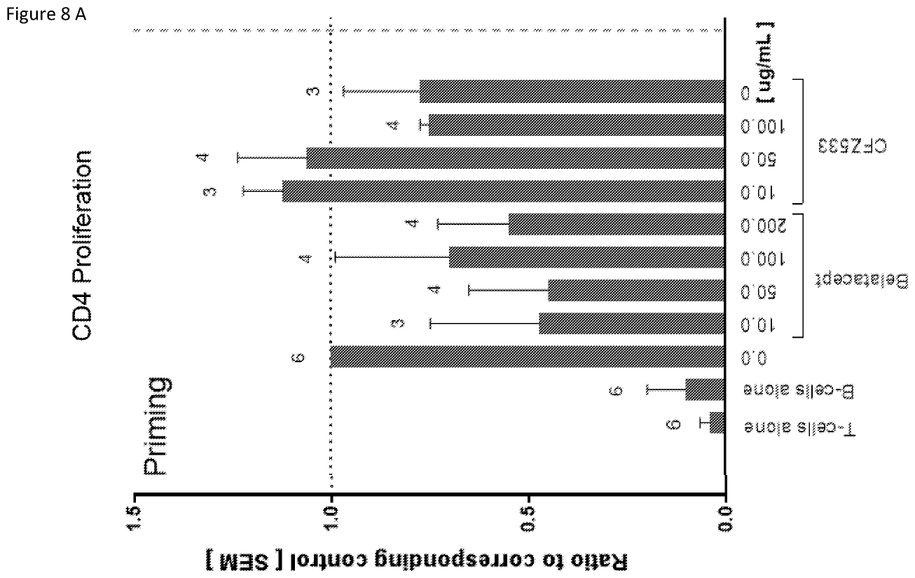

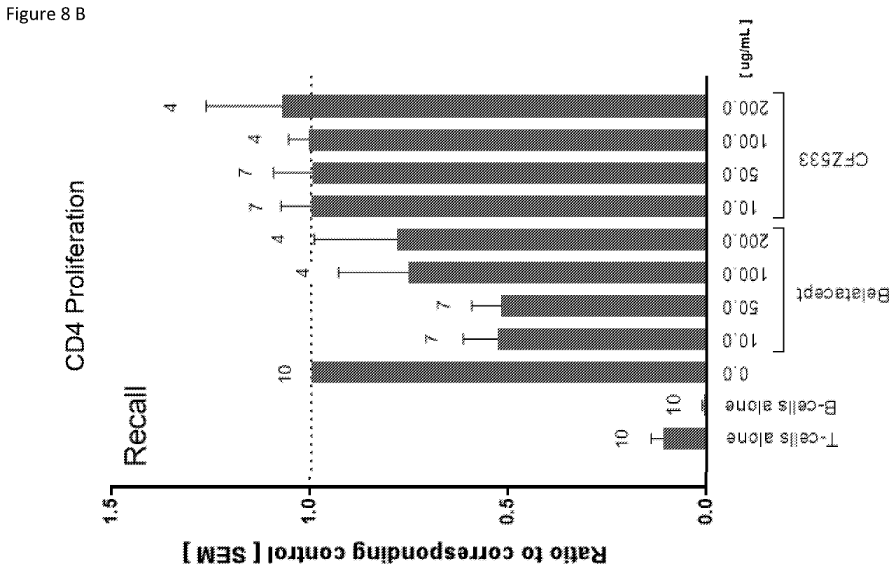

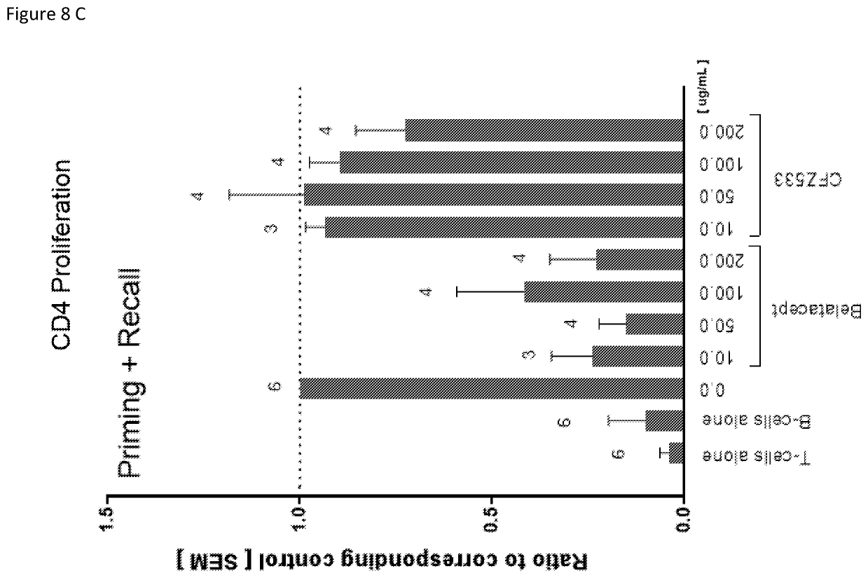

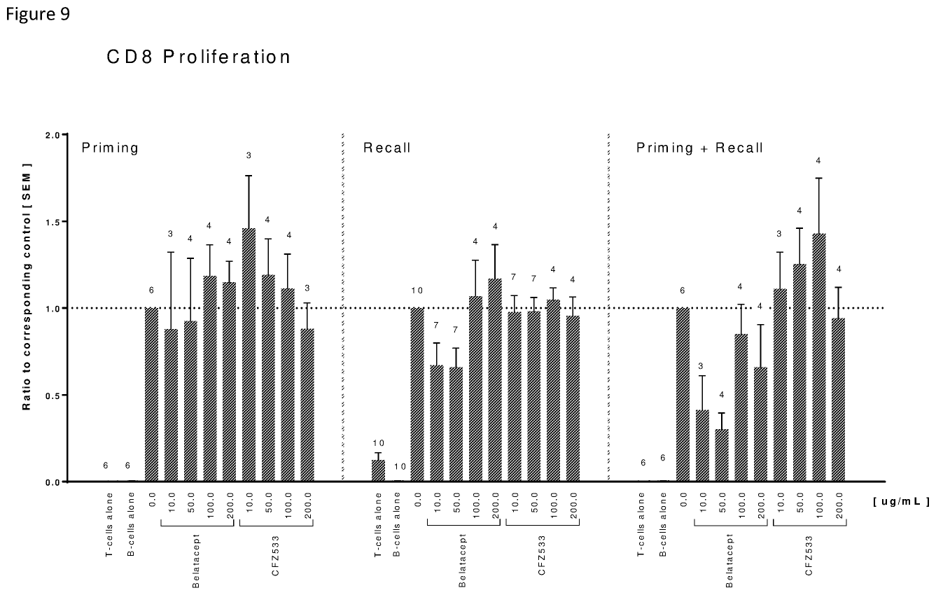

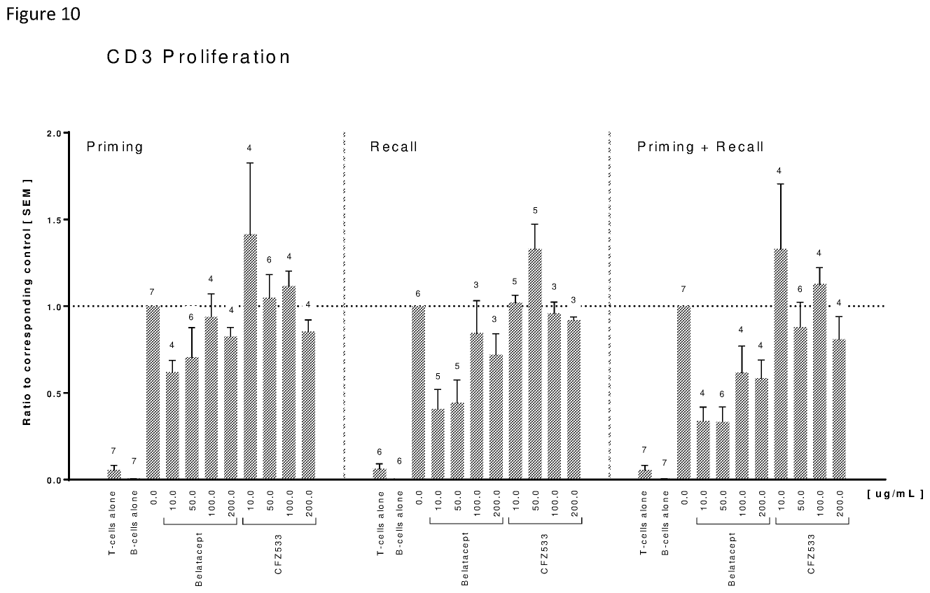

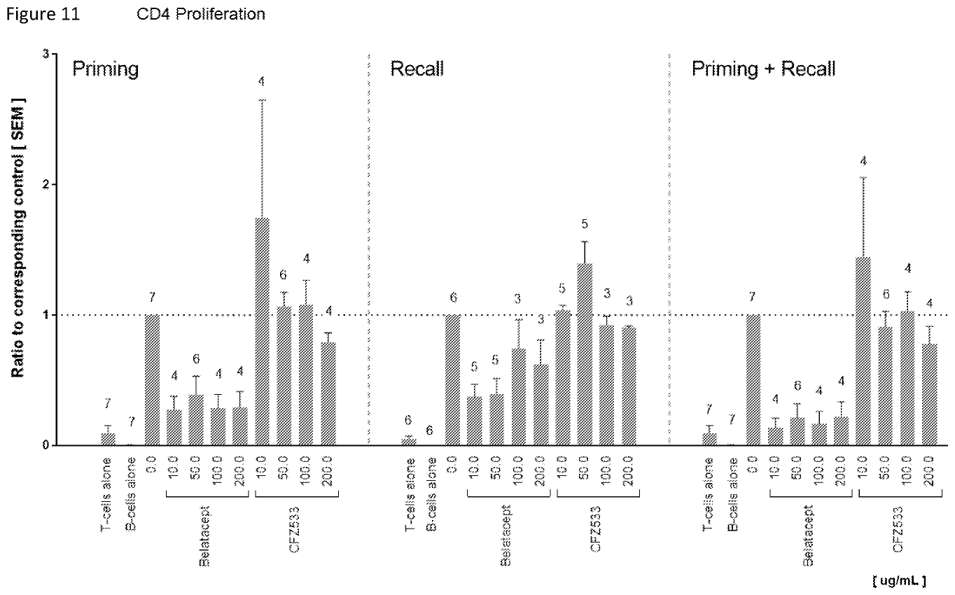

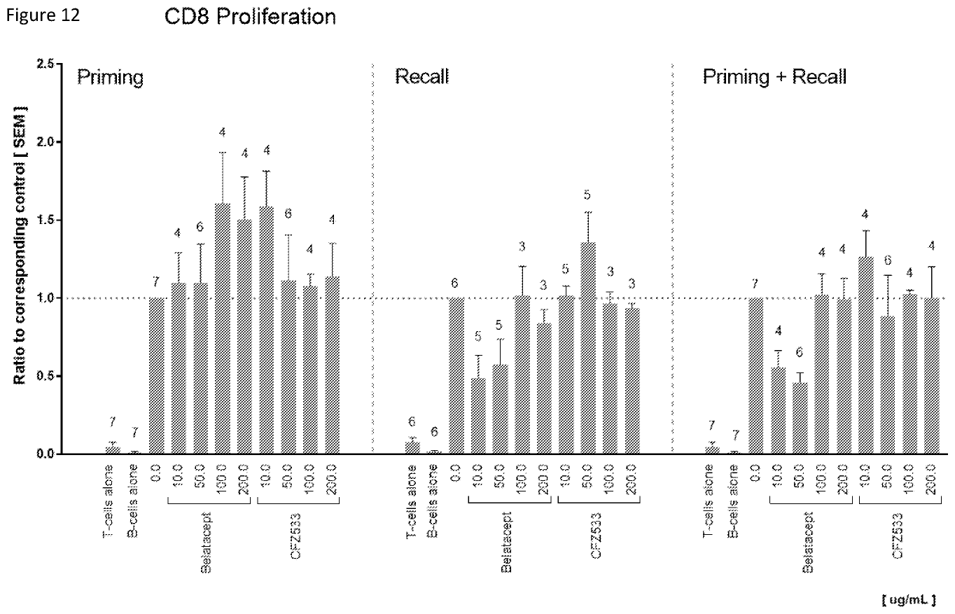

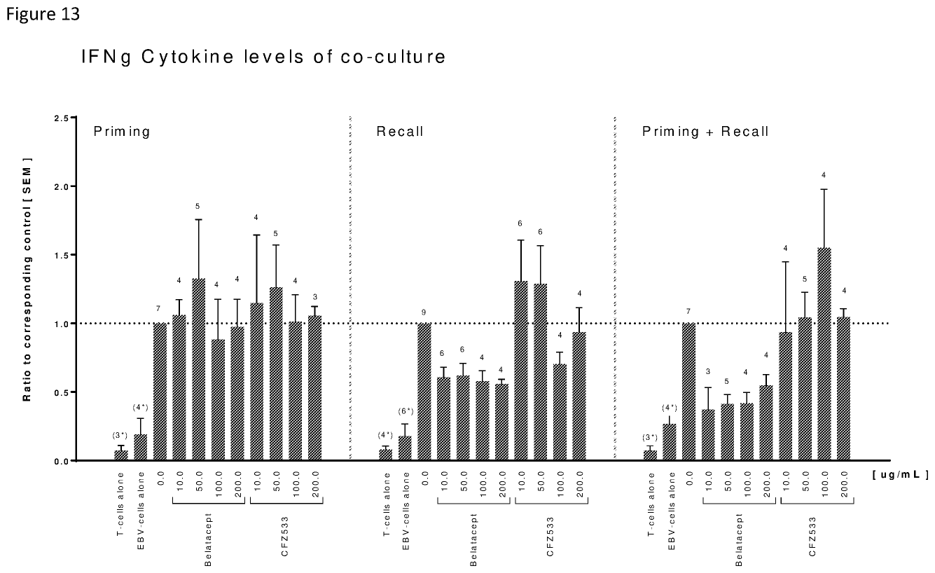

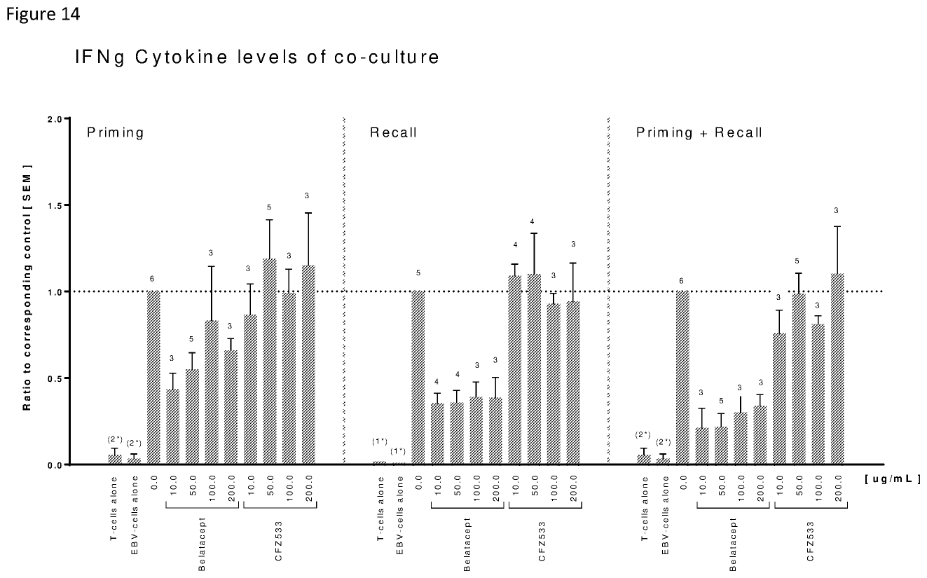

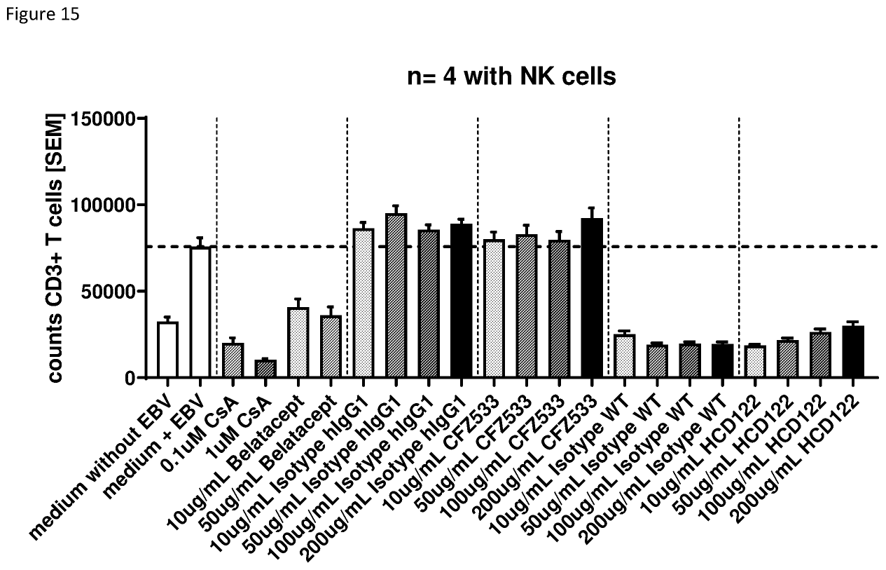

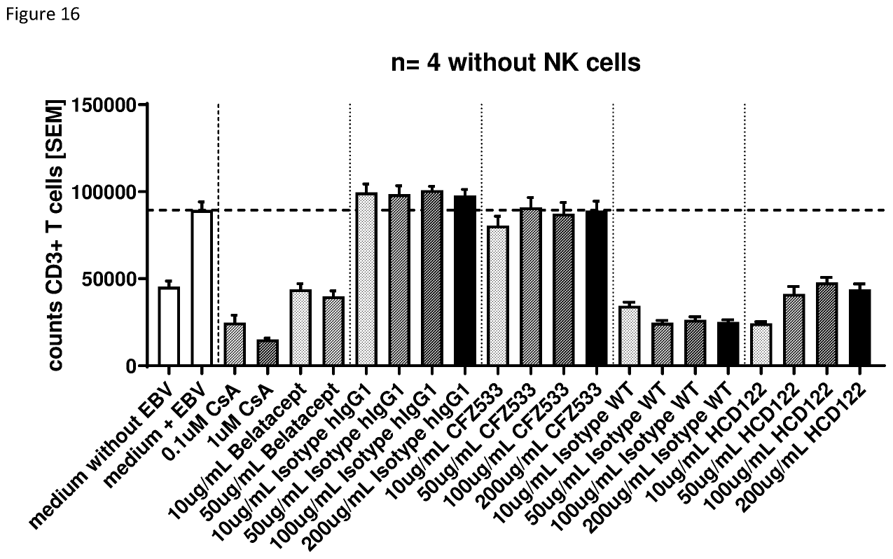

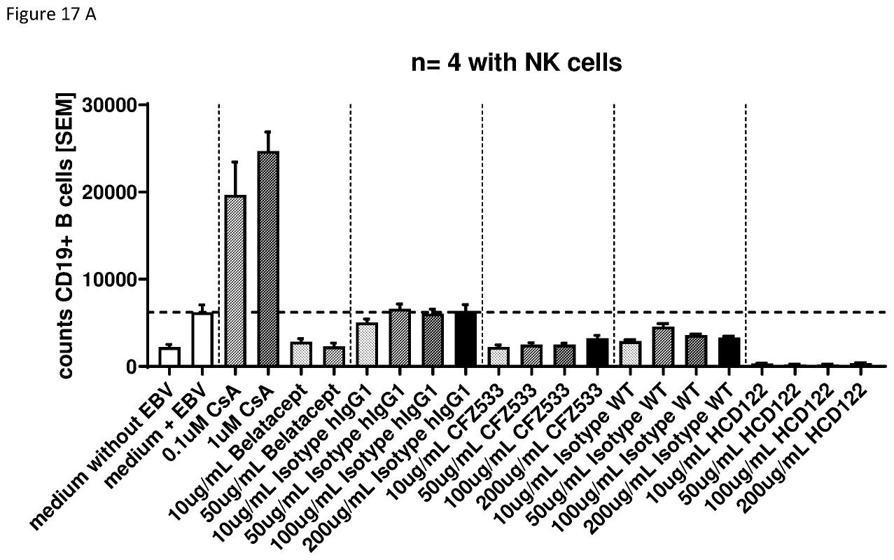

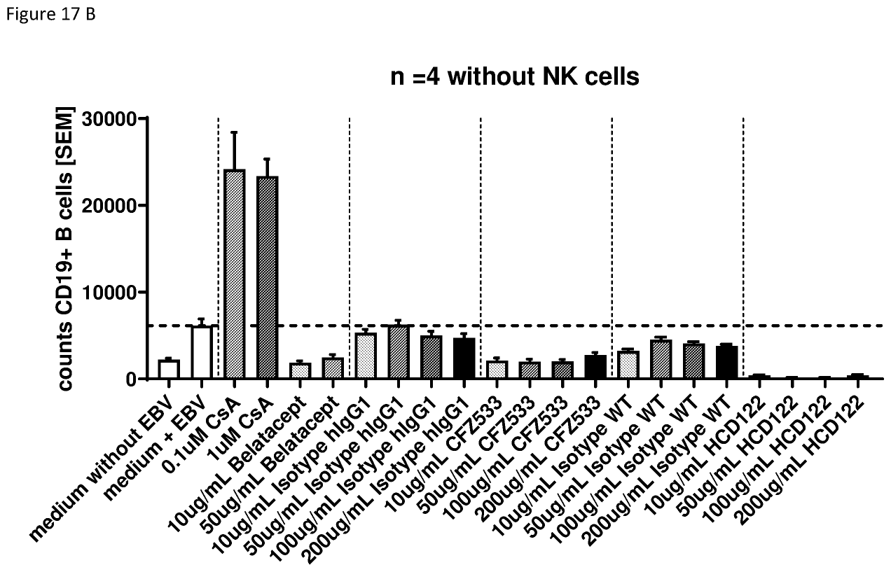

However, it will be understood by a skilled artisan that this naming convention is simply used for consistency and should not be construed as limiting, i.e., weekly dosing is the provision of a weekly dose of the anti-CD40 antibody, e.g., mAb1, regardless of whether the physician refers to a particular week as “week 1” or “week 2”. Example of dosage regimes as noted herein are found in FIGS. 1 and 2. It will be understood that a dose need not be provided at an exact time point, e.g., a dose due approximately on day 29 could be provided, e.g., on day 24 to day 34, e.g., day 30, as long as it is provided in the appropriate week.

As used herein, the phrase “container having a sufficient amount of the anti-CD40 antibody to allow delivery of [a designated dose]” is used to mean that a given container (e.g., vial, pen, syringe) has disposed therein a volume of an anti-CD40 antibody (e.g., as part of a pharmaceutical composition) that can be used to provide a desired dose. As an example, if a desired dose is 500 mg, then a clinician may use 2 ml from a container that contains an anti-CD40 antibody formulation with a concentration of 250 mg/ml, 1 ml from a container that contains an anti-CD40 antibody formulation with a concentration of 500 mg/ml, 0.5 ml from a container contains an anti-CD40 antibody formulation with a concentration of 1000 mg/ml, etc. In each such case, these containers have a sufficient amount of the anti-CD40 antibody to allow delivery of the desired 500 mg dose.

As used herein, the phrase “formulated at a dosage to allow [route of administration] delivery of [a designated dose]” is used to mean that a given pharmaceutical composition can be used to provide a desired dose of an anti-CD40 antibody, e.g., mAb1, via a designated route of administration (e.g., SC or IV). As an example, if a desired subcutaneous dose is 500 mg, then a clinician may use 2 ml of an anti-CD40 antibody formulation having a concentration of 250 mg/ml, 1 ml of an anti-CD40 antibody formulation having a concentration of 500 mg/ml, 0.5 ml of an anti-CD40 antibody formulation having a concentration of 1000 mg/ml, etc. In each such case, these anti-CD40 antibody formulations are at a concentration high enough to allow subcutaneous delivery of the anti-CD40 antibody. Subcutaneous delivery typically requires delivery of volumes of about 1 mL or more (e.g. 2 mL). However, higher volumes may be delivered over time using, e.g. a patch/pump mechanism.

Disclosed herein is the use of an anti-CD40 antibody (e.g., mAb1) for the manufacture of a medicament for preventing an EBV-associated disorder or for controlling EBV load in a patient, wherein the medicament is formulated to comprise containers, each container having a sufficient amount of the anti-CD40 antibody to allow delivery of at least about 75 mg, 150 mg, 300 mg or 600 mg anti-CD40 antibody or antigen binding fragment thereof (e.g., mAb1) per unit dose.

Disclosed herein is the use of an anti-CD40 antibody (e.g., mAb1) for the manufacture of a medicament for the prevention of an EBV-associated disorder or for controlling EBV load, wherein the medicament is formulated at a dosage to allow systemic delivery (e.g., IV or SC delivery) 75 mg, 150 mg, 300 mg of 600 mg anti-CD40 antibody or antigen binding fragment thereof (e.g., mAb1) per unit dose.

In one specific embodiment, a use is provided, of a) a liquid pharmaceutical composition comprising an anti-CD40 antibody, a buffer, a stabilizer and a solubilizer, and b) means for subcutaneously administering the anti-CD40 antibody to a transplantation patient, for the manufacture of a medicament for the prevention of an EBV-associated disorder or for controlling EBV load, wherein the anti-CD40 antibody:
i) is to be subcutaneously administered to the patient with a dose of about 3 to about 30 mg, such as 10 mg, active ingredient per kilogram of a human subject, three times, once every other week; and
ii) thereafter, is to be subcutaneously administered to the patient as monthly doses of about 3 to about 30 mg, such as 10 mg, active ingredient per kilogram of a human subject, wherein said anti-CD40 antibody is selected from the group consisting of:
a) an anti-CD40 antibody comprising an immunoglobulin VH domain comprising the amino acid sequence of SEQ ID NO: 7 and an immunoglobulin VL domain comprising the amino acid sequence of SEQ ID NO: 8;
b) an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
c) an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13;
d) an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14;
e) an anti-CD40 antibody comprising a silent Fc IgG1 region: and
f) an anti-CD40 antibody comprising the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

### Example 2. Pharmacology

### 1. Primary Pharmacology

mAb1 binds to human CD40 with high affinity (Kd of 0.3 nM). However, it does not bind to Fcγ receptors (including CD16) or mediate antibody-dependent cellular cytotoxicity or complement-dependent cytotoxicity. mAb1 inhibits recombinant CD154 (rCD154)-induced activation of human leukocytes, but does not induce PBMC proliferation or cytokine production by monocyte-derived dendritic cells (DCs). mAb1 binds human and non-human primate CD40 with very similar affinities.

In vivo, mAb1 blocks primary and secondary T cell-dependent antibody responses (TDAR), and can prolong survival of kidney allografts in non-human primates (Cordoba et al 2015). In addition, mAb1 can disrupt established germinal centers (GCs) in vivo. The CD40 receptor occupancy and functional activity were simultaneously assessed in vitro using human whole blood cultures. Functional activity was quantified via CD154-induced expression of CD69 (the activation marker) on CD20 positive cells (B cells) and CD40 occupancy was monitored using fluorescently labeled mAb1. Almost complete CD40 occupancy by mAb1 was required for full inhibition of rCD154-induced CD69 expression.

### 2. Secondary Pharmacology

The effects of mAb1 on platelet function and blood hemostasis were investigated, indicating that mAb1 does not induce platelet aggregation responses, rather displays certain mild inhibitory effects on platelet aggregation at high concentrations.

### Example 3. Non-Clinical Toxicology and Safety Pharmacology

Toxicology studies with mAb1 did not reveal any significant organ toxicities, including no evidence of thromboembolic events as reported in clinical trials with anti-CD154 mAbs (Kawai et al 2000). In a 13-week GLP rhesus monkey study (weekly dosing at 10, 50 and 150 mg/kg), increased lymphoid cellularity was noted in 5/22 animals which was considered to be due to ongoing infection, an observation consistent with the pharmacology of mAb1. Inflammatory lesions in the kidneys and lungs of 2 animals at 50 mg/kg were noted, and in one of the two animals, lesions in the eyes and trachea were also noted. While a direct effect of mAb1 on the kidney and lung cannot be excluded, the weight of evidence including confirmation of opportunistic pathogens, suggests these findings are likely secondary to mAb1-mediated immunosuppression and of an infectious origin. In view of these inflammatory findings, the No Observed Adverse Effect Level (NOAEL) for the 13-week toxicity study was set at 10 mg/kg. In a 26-week chronic toxicity study in cynomolgus monkeys, no adverse, mAb1-related findings were discovered. Based on these data, the NOAEL was set at 150 mg/kg (26-week). The mean (all animals) Cmax,ss was 44, 3235, and 9690 μg/mL at 1, 50, and 150 (NOAEL) mg/kg S.C. weekly, respectively. The NOAEL derived from the 26-week cynomolgus monkey study is considered the most relevant for supporting the clinical dosing regimen.

Post-mortem histological and immuno-histological evaluation revealed a decrease in GCs in cortical B-cell areas of the spleen and lymphatic tissues. The recovery animals showed some cases of increased lymph node cellularity with normal T cell areas and increased B cell areas, which is consistent with reconstitution of GCs after drug withdrawal. Recovery animals were able to mount primary TDAR to keyhole limpet hemocyanin (KLH) immediately after blood levels of mAb1 dropped below the level necessary for full receptor occupancy.

Because of the complete inhibition of T cell-dependent antibody responses (TDAR), KLH, the formation of anti-drug antibodies (ADA) to mAb1 is not expected and therefore ADA-related side effects are considered unlikely when concentrations of mAb1 are maintained continuously at pharmacological levels.

Tissue cross-reactivity studies revealed that CD40 is not only present on immune cells, but also in various tissues. This is mainly due to its expression on endothelial and epithelial cells, where CD40 is involved in signaling such as responding to wound healing processes, upregulation of virus-defense, and inflammatory-related mediators. An antagonistic anti-CD40 monoclonal antibody like mAb1 is not expected to contribute to inflammatory processes, which was confirmed by in vitro studies using human umbilical vein endothelial cells (HUVEC).

### Example 4. Non-Clinical Pharmacokinetics and Pharmacodynamics

### 1. Pharmacokinetics (PK)

Typical for IgG immunoglobulins, the primary route of elimination of mAb1 is likely via proteolytic catabolism, occurring at sites that are in equilibrium with plasma. In addition, binding and internalization of mAb1-CD40 complexes resulted in rapid and saturable clearance routes. This was illustrated by non-linear mAb1 serum concentration-time profiles showing an inflection point at about 10-20 μg/mL. The contribution of the CD40-mediated clearance to the overall clearance depends on mAb1 concentration, together with levels of CD40 expression, internalization and receptor turnover rates. For serum concentrations of mAb1 >10-20 μg/mL, linear kinetics are expected, while non-linear kinetics emerged at lower concentrations.

### 2. Pharmacodynamics (PD)

In a PK/PD study in cynomolgus monkeys, the inflection point (about 10 μg/mL) in the PK profiles was associated with a drop of CD40 saturation, as determined in an independent lymphocyte target saturation assay. As such this inflection point is viewed as a marker for the level of saturation of CD40, and an evidence for target engagement. The link between CD40 occupancy and pharmacodynamic activity was further demonstrated in rhesus monkeys immunized with KLH. Monkeys were immunized with KLH three times (the first was about 3 weeks prior to dosing, the second was 2 weeks after mAb1 administration, and the third was after complete wash-out of mAb1). CD40 occupancy by mAb1 at plasma concentrations >40 μg/mL at the time of the second KLH vaccination completely prevented recall antibody responses. Once mAb1 was cleared, all animals mounted a full memory antibody response to the third KLH. These results suggest that the function of preexisting memory B cells were not affected. After complete elimination of mAb1, immunization with tetanus toxoid (TTx) led to anti-TTx-IgG/IgM titers similar to non-treated animals and demonstrated that full TDAR was regained after mAb1 elimination.

### Example 5. Human Safety and Tolerability Data

The safety, tolerability, PK and PD activity of mAb1 are being assessed in an ongoing, randomized, double-blind, placebo-controlled, single-ascending dose study of mAb1 in healthy subjects and patients with rheumatoid arthritis (RA). A total of 48 subjects have been enrolled: 36 healthy subjects who received single doses of mAb1 up to 3 mg/kg IV or S.C., and 12 patients with RA, 6 of whom received single doses of mAb1 at 10 mg/kg IV. Overall, single doses up to 3 mg/kg mAb1 in healthy volunteers and a single of 10 mg/kg mAb1 in RA patients have been safe and well tolerated and no suspected serious adverse events (SAEs) have occurred. An investigation of the 30 mg/kg IV dose is ongoing in RA patients. As this study is still ongoing, all clinical data are preliminary in nature and based on interim analyses conducted up to a dose of 10 mg/kg in RA patients.

### Example 6. Human Pharmacokinetics and Pharmacodynamics (Healthy Volunteers and Rheumatoid Arthritis Patients)

In healthy subjects as well as in patients with rheumatoid arthritis, after single IV or SC administration, CFZ533 PK profiles were consistent with target mediated disposition resulting in non-linear PK profiles and more rapid clearance when CD40 receptor occupancy dropped below approximately 90%.

Despite some inter-individual variability in the PK profiles from the Chinese subjects, the disposition of CFZ533 in Chinese subjects was generally similar as for non-Chinese subjects, and the target engagement was also similar (about 4 weeks) after 3 mg/kg IV CFZ533. At this dose level, similar PK/PD profiles were demonstrated through free CFZ533 profiles in plasma, CD40 occupancy on peripheral B cells measuring free CD40 and total CD40, and total sCD40 concentrations in plasma.

After SC administration in healthy subjects, CFZ533 was rapidly absorbed and distributed in line with what is expected for a typical IgG1 antibody in human. At 3 mg/kg SC, CFZ533 generally peaked at 3 days post-dose (7 days for 2 subjects), and 1 week after dosing plasma concentrations were in the same range as for after IV. At 3 mg/kg SC, duration of target engagement was also about 4 weeks.

In patients with rheumatoid arthritis at 10 mg/kg IV, as measured by free CD40 on whole blood B cells compared to mean pre-dose, and total sCD40 profiles in plasma, full CD40 occupancy was generally maintained for 8 weeks. At 30 mg/kg IV, PK and total sCD40 profiles in plasma are consistent with duration of target engagement of 16 weeks.

In healthy subjects CD40 engagement by CFZ533 generally led to a decrease in total CD40 on peripheral B cells by about 50%, tracking CD40 occupancy on B cells as measured by free CD40 on B cells. This is likely due to internalization and/or shedding of the membrane bound CD40 upon binding to CFZ533. In patients with rheumatoid arthritis the decrease in total CD40 on peripheral B cells was not confirmed.

The relationship between CFZ533 in plasma and CD40 occupancy on whole blood B cells (free CD40 on B cells) was defined, and CFZ533 concentrations of 0.3-0.4 μg/mL were associated with full (defined as >90%) CD40 occupancy on whole blood B cells.

More generally, non-specific and specific elimination pathways have been identified for CFZ533. The non-specific and high capacity pathway mediated by FcRn receptors is commonly shared by endogenous IgGs. The specific target mediated disposition of CFZ533 led to the formation of CFZ533-CD40 complexes that were partially internalized (with subsequent lysosomal degradation) and/or shed from the membrane. Target-mediated processes resulted in saturable and nonlinear disposition of CFZ533. The formation of CFZ533-CD40 complexes was dose/concentration-dependent, with saturation occurring at high concentrations of CFZ533.

Overall, the disposition of CFZ533 is dependent on the relative contribution of the specific (target mediated) and non-specific elimination pathways to the overall clearance of CFZ533. Nonlinear PK behavior was observed when CFZ533 concentrations were lower than that of the target, while at higher concentrations with CD40 receptors being saturated, the non-specific pathways predominate and the elimination of CFZ533 was linear.

As expected for a typical IgG1 antibody targeting a membrane bound receptor and demonstrating target mediated disposition, the extent of exposure of CFZ533 (AUClast) increased more than the increase in dose (hyper-proportionality). Consequently, this is expected to be associated with a decrease in the volume of distribution and clearance of CFZ533 at higher doses.

A single dose of 3 mg/kg (IV and SC) of CFZ533 transiently suppressed anti-KLH responses to the first KLH immunization, at CFZ533 concentrations corresponding to full (>90%) receptor occupancy (for about 3-4 weeks). Anti-KLH primary responses were detected in all subjects as CFZ533 concentration, and accompanying receptor occupancy, declined. All subjects were able to mount recall responses to a second KLH immunization (administered after loss of receptor occupancy was anticipated).

Data suggest that CD40 engagement by CFZ533 prevented recombinant human CD154 (rCD154) mediated B cell activation in human whole blood. The rCD154-induced-CD69 expression on B cells was generally suppressed during a period corresponding to full CD40 occupancy on B cells. When CD40 occupancy was incomplete, the functional activity of rCD154 was restored.

Safety and tolerability confirmed in humans: A Phase 1 study (CCFZ533×2101), testing Single ascending doses (0.03 to 30 mg/kg) of CFZ533 i.v. and 3 mg/kg s.c., was completed and did not reveal major safety concern up to the highest dose tested (10 mg/kg i.v.). Based on clinical experience so far, the 10 mg/kg i.v. dosing regimen is anticipated to be safe and tolerable in transplant patients.

Adequate safety margin from preclinical toxicological studies: GLP toxicology studies to date have tested CFZ533 at (i) weekly s.c. dosing for 13 weeks at 10, 50, and 150 mg/kg (s.c. and i.v.) in rhesus monkeys, and (ii) weekly s.c. dosing for 26 weeks at 1, 50, and 150 mg/kg in cynomolgus monkeys. These studies did not reveal any major finding that would prevent the use of CFZ533 at the proposed intravenous regimen for 12 weeks or 24 weeks.

In the 26-week toxicity study in cynomolgus monkey, at steady state, an average concentration of 8300 μg/mL (Cav,ss) was obtained after weekly dosing at 150 mg/kg (NOAEL). The corresponding systemic exposure (AUC, steady state conditions) over a 1-month period would be 232400 day*μg/mL, which is about 57-fold higher than the predicted systemic plasma exposure over the first month (AUC0-28 days; FIG. 3). In the 26-week toxicology study, at NOAEL, Cmax, ss was 9495 μg/mL, which is 24-fold higher than the expected Cmax (about 400 μg/mL) for the proposed intravenous regimen in transplant patients (FIG. 4).

FIG. 4 shows predicted mean plasma concentration-time profile for CFZ533 given intravenously at 10 mg/kg (Cohort 2). Mean PK profiles were simulated for 10 mg/kg i.v. CFZ533 given at Study Day 1, 15, 29 and 57 (placebo controlled period), and Study Day 85, 99, 113 and 141 (open-label period). A Michaelis-Menten model was applied using parameters obtained from a preliminary model-based population analysis of Cohort 5 (3 mg/kg i.v.) PK data from FIH study CCFZ533×2101 in healthy subjects. No previous experience with an anti-CD40 blocking agent existed in human tx, and any potential differences in the biology of CD40 (expression, turnover) between healthy subjects and tx patients was no known. The proposed i.v. regimen was expected to provide, throughout the entire treatment period, sustained plasma concentrations above 40 μg/mL, to anticipate for an increased CD40 expression in target tissues in tx patients. The horizontal dotted line at 40 μg/mL is representing plasma concentration above which it is expected full CD40 occupancy and pathway blockade in target tissues (based on PD data from 26-week toxicology study in cynomolgus monkey—dose group 1 mg/kg). The expected systemic exposure for the first month (higher dosing frequency) is 4087 day*μg/mL (57-fold lower than the observed systemic plasma exposure over one month at steady state in the 26-week toxicology study in cynomolgus—NOAEL at 150 mg/kg weekly), the expected Cmax is about 400 μg/mL.

Relevant PD effects in tissues in non-human primates: In the 26-week toxicological study (1 mg/kg dose group) animals with average steady state plasma concentrations >38 μg/mL had a complete suppression of germinal centers in cortical B cell areas of lymph nodes. The 10 mg/kg i.v. regimen was expected to provide, throughout the entire treatment period (placebo-controlled and open-label, see FIG. 3), sustained plasma concentrations above 40 μg/mL, to anticipate for higher CD40 expression in tx patients, and incomplete PD effects in target tissue due to loss of target saturation.

### Material and Methods

TABLE 1
List of abbreviations

| Abbreviation | Description |
|---|---|
| ADCC | Antibody-dependent Cell-mediated Cytotoxicity |
| APC | Allophycocyanin |
| CD | Cluster of differentiation |
| CDC | Complement-dependent Cytotoxicity |
| CNI | Calcineurin inhibitors |
| CsA | Cyclosporine A |
| DPBS | Dulbecco's phosphate-buffered saline |
| EBV | Epstein-Barr-virus |
| EDTA | Disodium ethylenediaminetetraacetic acid |
| FBS | Fetal bovine serum |
| FSC | Forward Scatter |
| HTS | High-Throughput Sampler |
| IFNγ | Interferon gamma |
| NEAA | Non-Essential Amino Acid |
| PBMC | Peripheral blood mononuclear cell |
| PE | Phycoerythrin |
| Pen/Strep | Penicillin/Streptomycin |
| PTLD | post-transplant lymphoproliferative disorders |
| RT | Room temperature |
| SEM | Standard error of the mean |
| APC | Allophycocyanin |
| APC-Cy7 | Allophycocyanin-cyanine dye 7 |
| BSA | Bovine serum albumin |
| BV650 | Brilliant Violet 650 |
| CD | Cluster of differentiation |
| ADCC | Antibody-dependent Cell-mediated Cytotoxicity |
| APC | Allophycocyanin |
| CFSE | Carboxyfluorescein succinimidyl ester |
| CNI | Calcineurin inhibitors |
| DEPC | Diethylpyrocarbonate |
| DMSO | Dimethylsulfoxide |
| DPBS | Dulbecco's phosphate-buffered saline |
| EBV | Epstein-Barr-virus |
| EDTA | Disodium ethylenediaminetetraacetic acid |
| FACS | Fluorescence-activated cell sorting |
| FBS | Fetal bovine serum |
| FITC | Fluorescein isothiocyanate |
| IFNγ | Interferon gamma |
| min | Minutes |
| NaCl | Sodium chloride |
| NEAA | Non-Essential Amino Acid |
| PB | Pacific Blue |
| PBMC | Peripheral blood mononuclear cell |
| PBS | Phosphate-buffered saline |
| PE | Phycoerythrin |
| PE-Cy7 | Phycoerythrin-Cyanine 7 |
| Pen/Strep | Penicillin/Streptomycin |
| PTLD | post-transplant lymphoproliferative disorders |
| RT | Room temperature |

TABLE 2
List of reagents

| Reagent | Vendor |
|---|---|
| RPMI-1640 medium | Gibco |
| FBS | Gibco |
| FBS (Hyclone) | Hyclone |
| Pen/Strep | Sigma Aldrich |
| Kanamycin | Gibco |
| GlutaMax | Gibco |
| Sodium Pyruvate | Gibco |
| MEM Non-Essential Amino Acids | Gibco |
| β-Mercapto-ethanol | Gibco |
| DPBS (Mg−/Ca−) | Gibco |
| EDTA | Promega |
| Ficoll-Paque PLUS | GE Healthcare |
| Trypan Blue solution (0.4%) | Sigma Aldrich |
| Human serum Type AB (Male) | Sigma Aldrich |
| Transferrin | Merck & Cie (conc. 6 mg/mL) |
| Human EBV supernatant | Novartis Basel |
| Brilliant Stain Buffer | BD Pharmingen |
| Anti-Mouse Ig, k/Negative | BD Pharmingen |
| Control Compensation Particles | |
| Set | |
| FACS Sheath fluid Solution | BD Pharmingen |
| RPMI 1640 | Gibco/Life technologies |
| Hyclone Fetal Bovine Serum | Hyclone |
| (FBS) | |
| Penicillin/streptomycin | Sigma |
| Kanamycin sulfate | Gibco/Life technologies |
| NEAA | Gibco/Life technologies |
| Sodium pyruvate | Gibco/Life technologies |
| β-mercaptoethanol | Gibco/Life technologies |
| DNase I | Calbiochem |
| DPBS (Mg⁻/Ca⁻) | Gibco/Life technologies |
| Fetal Bovine Serum (FBS) | Gibco/Life technologies |
| Trypan Blue solution | Sigma-Aldrich |
| EasySep Human T Cell | Stemcell technologies |
| Enrichment Kit | |
| EasySep Human B Cell | Stemcell technologies |
| Enrichment Kit | |
| Human CD19 Microbeads Kit | Miltenyi Biotec |
| (MACS) | |
| BSA Stock solution MACS | Miltenyi Biotec |
| AutoMACS BSA Rinsing | Miltenyi Biotec |
| solution | |
| UltraPure DEPC Treated Water | Gibco/Life technologies |
| Recombinant human (rh) IL-2 | Novartis |
| EasySep Human B-Cell | Stemcell technologies |
| Enrichment Kit | |
| CellTrace CFSE Cell | Life Technologies |
| Proliferation Kit | |
| CellTrace Violet Cell | Life Technologies |
| Proliferation Kit | |
| IFNγ ELISA | Invitrogen/Thermo Fisher Scientific |
| DMSO | Sigma |
| Human Anti-Epstein Barr virus | Abcam |
| IgG Elisa Kit (EBV-EBNA) | |
| EDTA | Promega |
| EBV-supernatant | Lab Traggiai |
| Transferrin | Calbiochem |
| CpG2006 | Microsynth |

CpG2006 sequence: 5′-TCC ATG ACG TTC CTG ACG TT-3′; Human EBV supernatant was produced as described in Traggiai E. Methods Mol Biol. 2012; 901:161-70.

### Antibodies

TABLE 3
List of antibodies

| Antibody | Labeling | Vendor |
|---|---|---|
| CD3 (Clone UCHT1) | PE | eBioscience |
| Anti-CD4 | APC-Cy7 | Becton Dickinson |
| Anti-CD4 | FITC | eBioscience |
| Anti-CD4 | eFluor 450 (PB) | eBioscience |
| Anti-CD4 | PE-Cy7 | eBioscience |
| Anti-CD8 | ECD (Texas Red) | Beckman Coulter |
| Anti-CD19 | APC | eBioscience |
| Anti-CD20 | BV650 | Becton Dickinson |

### Compounds

TABLE 4
List of compounds

| | | Stock | | Final |
|---|---|---|---|---|
| | | concentration | | concentration |
| Compound | Vendor | (mg/mL) | | (μg/mL) |
| Belatacept | Novartis | 23.8 | | 50, 10 |
| CFZ533 | Novartis | 5.0 | | 200, 100, 50, 10 |
| IgG1 (Protrack | Novartis | 11.12 | | 200, 100, 50, 10 |
| No 107518; | | | | |
| Cyclosporine A | Tocris | 60.8 | (50 mM) | 24.2, 12.1, |
| (CsA) | (cat. 1101) | 121 | (100 mM) | 1.21, 0.121 |

| Compounds | Dissolved in | Stock concentration | |
|---|---|---|---|
| Belatacept | PBS, pH 7.4 | 23.8 | mg/mL |
| CFZ533 | PBS, pH 7.4 | 5 | mg/mL |
| IgG1 (isotype | 50 mM Citrate, 90 mM | 11.12 | mg/mL |
| for CFZ533) | NaCl, pH 7.0 | | |

Note:
CsA: 0.1 μM (=0.121 μg/mL), 1 μM (=1.21 μg/mL), 10 μM (=12.1 μg/mL), 20 μM (=24.2 μg/mL)

### Equipment and Supplementary Material

TABLE 5
List of equipment

| Equipment | Equipment name |
|---|---|
| Flow Cytometer | Fortessa X-20, BD Biosciences |
| BD HTS Reader | HTS, BD Biosciences |
| Centrifuges | Multifuge X3R Thermo Scientific 5810R Eppendorf |
| Sonicator | Ultrasonic cleaner, VWR |
| Vortexer | Vortex-Genie 2, Scientific Industries |

TABLE 6
Software

| Software | Version |
|---|---|
| BD FACSDiva Software | 8.0.2 |
| Flow Jo | 10 |
| GraphPad Prism | 7 |

### Medium, Buffers and Reagents

Cell culture medium: 425 mL RPMI-1640 medium, 5 mL sodium Pyruvate (final 1 mM), 5 mL Pen/Strep (1×), 5 mL Kanamycin (1×), 5 mL MEM-NEAA (final 1 mM), 5 mL L-Glutamine (final 2 mM), 0.5 mL β-mercaptoethanol (final 50 μM), 50 mL FBS (Hyclone; final 10%); EBV medium: 15 mL cell culture medium, 15 mL EBV-supernatant (final 25%), 0.6 mL Transferrin (final 30 ng/mL; stock 6 μg/mL); FACS-buffer: 500 mL DPBS, 10 mL FBS (final 2%), 2 mL EDTA (final 2 mM); Blocking buffer: 13.5 mL FACS-buffer, 1.5 mL human serum (final 10%).

B cell immortalization medium was done by mixing ‘complete medium’ (see above) with final concentration of 2.5 μg/mL CpG2006, 30 ng/mL Transferrin and 30% EBV-supernatant. In-house generated EBV-B cell lines and cell co-cultures were kept in RPMI-1640 medium supplemented with 10% Hyclone-FBS, 1× penicillin/streptomycin, 1× kanamycin, 1×NEAA, 1× sodium pyruvate and 50 μM β-mercaptoethanol (=complete medium). Freezing medium consisted of 10% DMSO and 90% FBS. MACS-buffer consisted of PBS supplemented with 0.5% BSA (MACS BSA stock solution was diluted 1:20 with autoMACS Rinsing solution) and 2 mM EDTA. 1 mg/mL DNAse was diluted with PBS to get a final concentration of 10 μg/mL (stock concentration), filtered through a 0.22 μm filter, aliquoted and stored at −20° C. 10 μL of the stock concentration was used per 1 mL complete medium. 1.2 mg lyophilized recombinant human (rh) IL-2 was diluted with 1.2 mL cell culture grade water (stock concentration: 1 mg/mL=18 MIO IU/1.2 mL=15 MIO IU/mL). Aliquots were made and stored at −20° C. For each experiment, fresh aliquots were thawed and keept at 4° C. for the duration of the experiment. One CFSE vial was diluted with 18 μL DMSO (stock concentration: 5 mM). For experimental procedures, a 1:5000 in PBS was used (final concentration: 1 μM). One Cell Trace Violet vial was diluted with 20 μL DMSO (stock concentration: 5 mM). For experimental procedures, a 1:2000 in PBS was used (final concentration: 2.5 μM).

Human donor selection from buffy coat: Buffy coats from 140 healthy volunteers were received from Blutspendezentrum Bern after signing the inform consent. Peripheral blood mononuclear cells (PBMC) and plasma from these donors were isolated and stored by NBC (NIBR), Basel, Switzerland. Before starting the experiments, all donors were analyzed by measuring IgG class antibodies against Epstein Barr virus (EBV) in plasma using the Human Anti-EBV IgG Elisa Kit (EBV-EBNA) following the manufacturer's instructions.

EBV-B cell line generation: Generation of EBV-B cell lines was done by using PBMC of the selected donors that were EBV-seropositive (first round: donor 88, 153 and 139; second round: donor 90, 125 and 171; third round: donor 633, 648, 638, 652, 637 and 660) and EBV-seronegative (first round: donor 73, 111 and 173; second round: donor 289 and 437; third round: donor 670, 624, 583 and 635).

First, B cells were purified from the selected PBMC, using the EasySep Human B cell Enrichment Cocktail following the manufacturer's protocol and adjusted to 1×106 cells/mL in undiluted EBV-supernatant (received from E. Traggiai, NBC, Novartis). After centrifugation (425×g, 3 h, 37° C.), supernatants of infected B cells were carefully removed and pellets were suspended in 1×106 cells/mL B cell immortalization medium (3.5). In parallel, feeder cells were prepared from non-autologous donors. Therefore, PBMC were isolated, irradiated at 50 Gray and kept at 4° C. until use. After centrifugation (425×g, 10 min, 4° C.), supernatants were carefully removed and cells were adjusted to 1×106 cells/mL in complete medium. 1 mL feeder cells and 1 mL infected B cells were distributed per well in a 12-well plate (final 2×106 cells/well/2 mL). Then, the cells were cultivated at 37° C., 5% CO2. For expansion, cells were splitted 1:2 with complete medium. Cells were transferred first into 6-well plates and later into T-75 flasks when the medium became yellow. Infected EBV-B cells were frozen for further use with freezing medium.

Co-culture of EBV-B cells or B cells and T cells: To establish an EBV-B cell/T cell co-culture model in-house for our investigations, we modified the protocol published by Andorsky et al. (Andorsky 2011). Here, we used EBV-B cells or primary B cells and autologous T cells, and incubated the cells with either an IgG1 isotype, anti-CD40 (CFZ533) or an anti-CTL-A4 antibody (Belatacept) at four different concentrations (10, 50, 100 and 200 μg/mL or vehicle control). The co-culture model consisted of two phases: ‘priming’ phase (seven days of culture) and ‘recall’ phase (additional four days of culture). The antibodies were added either at the ‘priming’ phase, ‘recall’ phase or at both phases (‘priming and recall’). After 11 days of co-culture, cells were analyzed by flow cytometry for T cell proliferation and by ELISA for IFNγ production.

Thawing of EBV-B cell lines: EBV-B cells were generated and frozen after successful expansion. One to two weeks before start of the experiment, frozen vial were thawed by gentle agitation in a 37° C. water bath or in hands. As soon as the content was thawed, the outer surface of the vial was decontaminated by using 70% ethanol. Thawed cells were transferred in a 15 mL Falcon tube and slowly added 3 mL of complete medium containing DNase (final 10 μg/mL). Afterwards, cells were centrifuged (365×g, 5 min, RT), supernatants were discarded and 6 mL complete medium added. Then, 6 mL of cells were put in one well of a 6-well plate and cultured at 37° C., 5% CO2. Cultures were maintained by adding fresh medium or by passaging in several wells of a 6-well plate or by replacing the medium every 2-3 days when the media turned orange-yellow. For the experiments, EBV-B cells were analyzed for CD3, CD19 and CD20 surface expression by flow cytometry.

Isolation of total B cells by positive selection: In parallel to EBV-B cells, also primary B cells were used in the experimental setup. Therefore, CD19 positive B cells were isolated by using the Human CD19 Microbeads Kit according to the manufacturer's instructions using the magnetic Auto MACS Separator. After the isolation, cells were centrifuged (300×g, 5 min, RT), supernatants were discarded and pellets were resuspended in complete medium. Then B cells were counted, kept in a T-25 flask at 4° C. and were irradiated.

Irradiation of EBV-B cells and primary B cells: EBV-B cells and isolated primary B cells kept in T-25 flasks were irradiated all at the same time at 30 Gray with an X-Ray RS-2000 irradiator. Irradiation will prevent primary B cells and EBV-B cells from dividing in these co-cultures. This will ensure that only T cells are dividing in response to stimulation and the APC (here primary B cells or EBV-B cells) will present the antigen and will die after several days in culture.

Isolation of total T cells by positive selection: With the unlabeled cells from the B cell isolation, T cells were isolated using EasySep Human T cell enrichment kit according to the manufacturer's instructions. After the isolation, cells were centrifuged (300×g, 5 min, RT), supernatants discarded and pellets were resuspended in complete medium. Afterwards, T cells were counted and stored at 4° C. until use.

Priming of T cells for 7 days: Isolated T cells (2×105/50 μL/well) were dispersed in 96-well U-bottom plates in complete medium containing 10 IU/mL rhIL-2. Afterwards, 2×104 irradiated EBV-B cells or primary B cells (50 μL/well) were added to the respective wells. Negative control conditions (B cells only, EBV-B cells only and T cells only) were filled with 50 μL complete medium. Then, 100 μL of the different antibodies and concentrations (see below) were put on top of the cells and incubated at 37° C., 5% CO2 for seven days where every two days, 150 μL old medium was carefully removed and 150 μL fresh complete medium supplemented with 10 IU/mL rhIL-2 was added on top. Concentrations used for IgG1, belatacept and CFZ533 were 10, 50, 100 and 200 μg/mL or vehicle control.

CellTrace CFSE and CellTrace Violet labeling: First, PBMC from the matching donors were thawed and primary B cells were isolated by negative selection using EasySep Human B cell enrichment kit according to the manufacturer's instructions. Then, primary B cells and maintained EBV-B cell cultures were labelled with CellTrace CFSE reagent. Briefly, cells were centrifuged (365×g, 7 min, RT), supernatants discarded, pellets were resuspended in 5 mL PBS/1% FBS and centrifuged (365×g, 7 min, RT). Then, cells were resuspended in 2 mL PBS/1% FBS and 2 mL of 1 μM CellTrace CFSE reagent diluted in PBS (final concentration 0.5 μM) was added to the cell and incubated (8 min, 37° C. in the dark). Afterwards, an equivalent volume (here: 4 mL) of pre-warmed FBS was added to quench the fluorescence and mixed gently. Cells were centrifuged (365×g, 10 min, RT), supernatants discarded, pellets resuspended in 5 mL complete medium and centrifuged (365×g, 10 min, RT). Then, pellets were resuspended in 4 mL complete medium, incubated (1 h, 37° C., in the dark), filled up with 6 mL complete medium. Cells were again centrifuged (365×g, 10 min, RT), supernatants discarded and pellets were resuspended in 5 mL complete medium. After counting, stained isolated primary B cells and EBV-B cells were put into T-25 flasks and irradiated as described. In addition, a small aliquot was taken and cells were analyzed by flow cytometry for successful CFSE labeling.

The same was done for cultured T cells. After seven days of culture, all cells were transferred in a sterile 96-well V-bottom plate, centrifuged (1000×g, 5 min, RT) and pellets were resuspended in 200 μL complete medium. 5 μL from each condition was taken and analyzed by flow cytometry for CD3, CD4, CD8, CD19 and CD20 expression and successful CFSE staining. Remaining cells were centrifuged (1000×g, 5 min, RT), supernatants discarded, pellets resuspended in 100 μL of 2.5 μM CellTrace Violet reagent and incubated (20 min, 37° C.). Then, cells were centrifuged (365×g, 10 min, RT), supernatants discarded, 100 μL of pre-warmed FBS was added to quench the fluorescence and mixed gently. Afterwards, cells were incubated (30 min, 37° C.), 150 μL of complete medium added, centrifuged (365×g, 10 min, RT), supernatants discarded and pellets were resuspended in 200 μL complete medium. All cells were counted and an aliquot from each donor was taken to check the successful CFSE staining by FACS.

Recall of co-culture for 4 days: For all conditions, 96-well U-bottom plates in duplicates were used. 2×104 CellTrace Violet labelled T cells (50 μL/well) were added to the respective wells and 2×104 irradiated and CellTrace CFSE-labelled EBV-B cells or primary B cells (50 μL/well) added on top. Then, 100 μL complete medium containing different concentrations of antibodies (see 4.3.5) were added and cultured (37° C., 5% CO2) for 4 days.

### Regression Assay

Buffy coats from six healthy volunteers (18-001 to 18-006) were received from Blutspendezentrum Bern. Peripheral blood mononuclear cells (PBMC) were isolated using LeucoSep tubes following manufacturer's instructions, counted with TC20 and adjusted in cell culture medium (Medium, buffers and reagents). All conditions (see Table 3-2) were run in five replicates in 96-well U-bottom plates and contained 2×105 cells/well. PBMC were incubated for 10-15 minutes at 37° C. before adding cell culture medium containing EBV-supernatant and transferrin (see Medium, buffers and reagents). Belatacept was tested in two different concentrations (10 μg/mL, 50 μg/mL), isotype hIgG and CFZ533 in four different concentrations (10 μg/mL, 50 μg/mL, 100 μg/mL, 200 μg/mL) and CsA in four different concentrations (0.1 μM, 1 μM, 10 μM, 20 μM). The plates were incubated for 14 days (37° C., 5% CO2) without adding any supplements. After 14 days, cells were transferred to a 96-well V-bottom plate, centrifuged at 1000×g for 5 minutes at RT and the obtained supernatants (200 μL) were stored at −80° C. for further analyses. Cell pellets were stained and analyzed by flow cytometry (FACS staining procedure).

### FACS Staining Procedure

Cell pellets from regression assay were washed with FACS-buffer and blocked with blocking buffer for 20 minutes at 4° C. Without washing, an antibody cocktail consisting of CD3-PE and CD19-APC was added and incubated for 20 minutes at 4° C. After the incubation, cells were washed twice with FACS-buffer, resuspended in 100 μL FACS buffer and 65 μL of cell suspension was acquired in plates (HTS reader) with the FortessaX20 and analyzed using FlowJo software. At different time points, cells were analyzed by flow cytometry for surface expression markers, like CD3, CD4, CD8, CD19 and CD20, and successful CellTrace CFSE or CellTrace Violet labelling. Furthermore, at the end of the co-culture time, cells were analyzed for T cell proliferation using a viability marker, CD3, CD4 and CD8. Therefore, cells were taken, placed in a 96-well V-bottom plate and washed with 200 μL FACS-buffer. Then, cells were centrifuged (1000×g, 5 min, RT) and stained with different antibodies (single for compensation or different antibody cocktails) for 30 min at 4° C. After two washing and centrifugation steps, cells were resuspended with 50 μL FACS-buffer, transferred to Micronics tubes and acquired with the BD LSRFortessa™ cell analyzer.

### Results:

Human donor selection: Before starting the co-cultures, we tested the EBV serotype using an ELISA as described before. We identified ten EBV-seropositive donors (donor 88, 90, 125, 139, 153, 171, 633, 637, 652 and 660) and eight EBV-seronegative donors (donor 73, 111, 289, 437, 583, 624, 635 and 670). From these donors, EBV-B cell lines were generated as described above, different cells isolated and used in the co-culture assay.

### FACS Results I.

For the cell co-culture model, we used EBV-B cells or primary B cells and autologous T cells, and incubated the cells with either an IgG1 isotype, anti-CD40 (CFZ533) or an anti-CTL-A4 antibody (Belatacept) at four different concentrations (10, 50, 100 and 200 μg/mL or vehicle control). Antibodies of interest were added at either the ‘priming’, ‘recall’ or ‘priming and recall’ phase to the cells as described before. After 11 days of co-culture, cells were analyzed by flow cytometry for T cell proliferation and by ELISA for IFNγ production.

In the experiments where co-cultures consisted of primary B cells and autologous T cells, the cell viability and proliferation or cell cluster formation observed microscopically and acquired by flow cytometry were low (EBV-seropositive and EBV-seronegative). Consequently, the results were inconclusive and are not shown in this report. Furthermore, this was also seen in the IFNγ cytokine levels where all conditions were under detection limit. Therefore, T cell proliferation (FIG. 7 to FIG. 12) and IFNγ cytokine level results (FIG. 13 and FIG. 14) will be shown only for co-cultures containing EBV-B cells and autologous T cells

### FACS Results II.

In total, we used and analyzed six different donors. We removed the two highest CsA concentrations (10 μM and 20 μM) from the analyses, as they were toxic to the cells. In addition, two donors (18-003 and 18-005) were excluded from the analyses, as CsA—that served as our positive control—did not show any effect on T and B cell proliferation.

For the other four donors, analyses were performed by using the described gating strategy (Gating strategy for CD3+ T cell and CD19+ B cell analyses and FIG. 1) and results are shown in FIG. 2 and FIG. 3.

Gating strategy for T cell proliferation: After 11 days of co-culture, cells were stained for CD3, CD4, CD8, CD19 and CD20 including a viability marker and analyzed by flow cytometry for T and B cell proliferation. In FIG. 6-1, the gating strategy for T cell proliferation is shown using EBV-B cell/T cell co-cultures. First, we plotted all acquired cells on the forward side scatter (FCS)/sideward scatter (SSC) in the dot plot and then gated on the total viable cells (viability marker) in a histogram plot. From the alive cell gate, CD3+ T cells were chosen and T cell proliferation was identified by gating on Cell Tracer Violet-positive cells (first row from left to right). Then, from the total viable cells, a quadrant was placed to identify CD4+ and CD8+ T cells and analyzed for proliferation having Cell Tracer Violet labelling (second row).

### Gating Strategy for CD3+ T Cell and CD19+ B Cell Analyses.

After 14 days, cells were stained with CD3 and CD19 antibodies, and analyzed by flow cytometry for CD3+ T cells and CD19+ B cells. In FIG. 1, the gating strategy is shown. First, we plotted all acquired cells on the forward scatter (FCS)/side scatter (SSC) in the dot plot and then further gated either on CD3+ T cells (upper purple circles) or CD19+ B cells (right purple circles) in the dot plot. Different conditions are shown from one representative donor ((first row: PBMC+medium-EBV (left plots), PBMC+medium+EBV (right plots); second row: PBMC+0.1 μM CsA+EBV (left plots), PBMC+50 μg/mL Belatacept+EBV (right plots); third row: PBMC+50 μg/mL hIgG1+EBV (left plots), PBMC+50 μg/mL CFZ533+EBV (right plots)).

T cell proliferation of EBV-seropositive EBV-B cell/T cell co-cultures: The overall design of the in vitro studies is depicted in FIG. 6. To investigate the T cell proliferation of total CD3+ T cells (FIG. 7), CD4+ T cells (FIG. 8) and CD8+ T cells (FIG. 9), different concentration (0, 10, 50, 100 or 200 μg/mL) of CFZ533 or Belatacept were given to the EBV-seropositive EBV-B cell/T cell co-cultures at indicated phases (‘priming’, ‘recall’, ‘priming and recall’) and analyzed as ratio to corresponding controls, like medium or isotype control. In all three experimental conditions, EBV-B cells induced CD3+, CD4+ and CD8+ T cell proliferation (indicated in the graphs as ‘0 μg/mL’) compared to T cells alone conditions. Belatacept reduced the EBV-driven T cell proliferation in presence of EBV-B cells strongest when the antibody was given twice to the co-culture (‘priming and recall’ phase) compared to the ‘priming’ and ‘recall’ only condition. Moreover, 10 and 50 μg/mL Belatacept were showing a slightly stronger but not significant suppression pattern than 100 and 200 μg/mL Belatacept in all three conditions. Additionally, 100 and 200 μg/mL belatacept had only an reducing effect on the T cell proliferation at the ‘priming and recall’ phase, but not when added only at the ‘priming’ or ‘recall’ phase. In contrast, CFZ533 had no reducing effect on the CD3+ T cell proliferation at all four tested concentrations.

Specifically analyzing the CD4+ T cell proliferation (FIG. 8), Belatacept also reduced the EBV-driven T cell proliferation in presence of EBV-B cells strongest when the antibody was given at ‘priming and recall’ phase compared to the ‘priming’ and ‘recall’ only condition. Additionally, 10 and 50 μg/mL Belatacept were inducing a slightly stronger but not significant suppression pattern on CD4+ T cell proliferation than 100 and 200 μg/mL Belatacept in all three conditions. Moreover, 100 and 200 μg/mL Belatacept had a robust reducing effect on the T cell proliferation at the ‘priming and recall’ phase, and to a lower extent on the ‘priming’ or ‘recall’ only phase. Interestingly, both high doses were still able to reduce T cell proliferation in contrast to CD3+ T cell proliferation results. Again as seen before, CFZ533 had no decreasing effect on the CD4+ T cell proliferation at all four tested concentrations except a slight decline in the ‘priming’ only phase.

Analyzing CD8+ T cell proliferation (FIG. 9), Belatacept and CFZ533 had similar effects as seen for the CD3+ T cell proliferation before (FIG. 7). T cell proliferation of EBV-seronegative EBV-B cell/T cell co-cultures The same approach was used to analyze T cell proliferation of total CD3+ T cells (FIG. 10), CD4+ T cells (FIG. 11) and CD8+ T cells (FIG. 12) from EBV-seronegative donors. As for the EBV-seropositive donors, in all three conditions, EBV-B cells induced CD3+, CD4+ and CD8+ T cell proliferation (indicated in the graphs as ‘0 μg/mL’) compared to T cells alone conditions. For CD3+ T cell proliferation (FIG. 10), Belatacept reduced the EBV-driven T cell proliferation in presence of EBV-B cells strongest when the antibody was given twice to the co-culture (‘priming and recall’ phase) compared to the ‘priming’ and ‘recall’ only condition. Furthermore, 10 and 50 μg/mL belatacept were showing a slightly stronger but not significant suppression pattern than 100 and 200 μg/mL Belatacept in all three conditions. Additionally, 100 and 200 μg/mL Belatacept had only a reducing effect on the T cell proliferation at the ‘priming and recall’ condition, and only minor at the ‘priming’ or ‘recall’ only condition. In contrast, CFZ533 had no reducing effect on the CD3+ T cell proliferation at all four tested concentrations.

Specifically evaluating CD4+ T cell proliferation (FIG. 11), Belatacept reduced the EBV-driven T cell proliferation in presence of EBV-B cells similarly strong at all three different conditions and stronger than in EBV-seropositive donors. Interestingly, no evident differences were seen between the four doses (10, 50, 100 and 200 μg/mL) as observed with EBV-seropositive donors. In contrast, CFZ533 had no effects on the CD4+ T cell proliferation except a slight not significant increase at 10 μg/mL (‘priming’ only and ‘priming and recall’ condition) and 50 μg/mL (‘recall’ only condition).

Specifically evaluating CD4+ T cell proliferation (FIG. 11), Belatacept reduced the EBV-driven T cell proliferation in presence of EBV-B cells similarly strong at all three different conditions and stronger than in EBV-seropositive donors. Interestingly, no evident differences were seen between the four doses (10, 50, 100 and 200 μg/mL) as observed with EBV-seropositive donors. In contrast, CFZ533 had no effects on the CD4+ T cell proliferation except a slight not significant increase at 10 μg/mL (‘priming’ only and ‘priming and recall’ condition) and 50 μg/mL (‘recall’ only condition).

### Effects on CD3+ T Cell and CD19+ B Cell Proliferation Using Different Stimuli.

To investigate the proliferation of total CD3+ T cells (FIG. 2) and CD19+ B cells (FIG. 3), different concentrations of hIgG1 or CFZ533 (10 μg/mL, 50 μg/mL, 100 μg/mL or 200 μg/mL), Belatacept (10 μg/mL or 50 μg/mL) or CsA (0.1 μM or 1 μM) were used. As already mentioned above, 10 μM and 20 μM CsA were toxic to the cells and were therefore excluded from the analyses. All conditions except ‘medium w/o EBV’ were incubated with EBV supernatant and respective compound or antibodies. As shown in FIG. 2, there was a clear increase in CD3+ T cell counts when PBMC were incubated with EBV supernatant compared to PBMC incubated with medium only (medium w/o EBV). In addition, there was a robust decease in CD3+ T cell counts with 0.1 μM and 1 μM CsA as expected as it served as our positive control compared to the control condition (medium+EBV). The same could be observed with Belatacept (10 μg/mL and 50 μg/mL) but to a lesser degree than with CsA. CFZ533 and the corresponding isotype control hIgG1 did not had any effect on CD3+ T cell counts for all four tested concentrations. Looking at the CD19+ B cell proliferation (FIG. 3), there was an obvious increase in CD19+ B cell counts when PBMC were incubated with EBV supernatant compared to PBMC cultured with medium only (medium w/o EBV). Furthermore, there was a strong increase in CD19+ B cell counts with 0.1 μM and 1 μM CsA as anticipated compared to the control condition (medium+EBV). Belatacept (10 μg/mL and 50 μg/mL) led to an increase in CD19+ B cell counts whereas isotype control hIgG1 did not had any effect for all four tested concentrations. In contrast, CFZ533 showed a decrease in CD19+ B cell counts probably due to an indirect NK cell-mediated antibody-dependent cellular cytotoxicity (ADCC) after 14 days. In the publication of Ristov et al. (Ristov 2018), no ADCC was seen in whole blood cultures incubated with CFZ533 for three days and in the publication of Cordoba et al. (Cordoba 2015), CFZ533 revealed no significant peripheral B cell depletion over the duration of the experiment (100 days post-kidney transplant in non-human primates, 13- and 26-week tox studies). The difference observed in terms of CD19 decrease could be explained by the different experimental setting: in the EBV regression assay, NK cells are clearly activated by EBV and the duration of the experiment allows their expansion, while in a three-day assays without any specific activation, the level of ADDC is neglectable. To investigate the potential involvement of NK cells, we performed a new set of experiments where NK cells were either kept or depleted in the cell culture model. In addition, we included HCD122, a depleting CD40 mAb as positive control. Similar results were obtained when analyzing CD19+ B cell counts in complete or NK cell-depleted PBMC cultures. HCD122 did deplete B cells as expected. Again, CFZ533 showed a decrease in CD19+ B cell counts compared to isotype control independently of NK cells in the PBMC cultures. Therefore, we can exclude NK cell-mediated ADCC. Indeed it has been shown that CD40-CD40L interaction is critical for the establishment of EBV-B cell lymphoma in vivo (Ma et al., 2015).

Epstein Barr Virus (EBV)-associated post-transplant iymphoproliferative disorders (PTLD) is linked to EBV primary infection or reactivation. In an immunocompetent individual, the anti-viral T cell response controls the infection but EBV remains latent in B cells and some other cell types. In transplanted patients, immunosuppression could dampen the anti-EBV T cell response, leaving EBV-induced B cell proliferation uncontrolled. The inventors examined the effects of an anti-CD40 mAb on T cell-driven control of EBV-B cells and consequently B cell outgrowth. To do so the inventors performed an EBV regression assay using peripheral blood mononuclear cells incubated with Cyclosporine A (CsA), CTLA4-Ig fusion protein (Belatacept) in comparison to the anti-CD40 mAb (CFZ533/Iscalimab). In addition, the inventors evaluated the effect of blocking CD40 or CTLA-4 on T cell proliferation and IFN production by using autologous co-cultures of T cells with EBV-B cells (sero-positive and sero-negative) or primary B cells. Belatacept and CsA but not anti-CD40 mAb Iscalimab reduced T cell activity resulting in over-growth of in vitro immortalized cells. Furthermore, Belatacept but not Iscalimab reduced EBV-mediated T cell proliferation and IFNγ secretion in presence of EBV-B cells using the co-culture system. In conclusion, Iscalimab does not impair EBV control in vitro, in contrast to CsA and Belatacept, suggesting that transplant patients dosed with Iscalimab may have a reduced risk of PTLD. In the EBV B cell/T cell co-culture model, Belatacept but not CFZ533/Iscalimab reduced EBV-mediated T cell proliferation and IFNγ secretion in presence of EBV-B cells. The inhibitory effects of Belatacept were most noticeable in the combined “priming” and “recall” cultures and on CD4+ T cells.

Belatacept and CsA but not CFZ533/Iscalimab reduced T cell activity resulting in over-growth of in vitro immortalized B cells. These results show that iscalimab has an improved safety profile in kidney organ transplant recipients compared to Belatacept. CFZ533/Iscalimab does not impair EBV control in vitro, in contrast to Belatacept, suggesting that transplant patients dosed with CFZ533/Iscalimab have a reduced risk of PTLD. Similar conclusions could be drawn for CsA/CNIs based on the EBV regression assay. These results appear consistent with the current clinical kidney transplantation data using Iscalimab as a CNI-free therapy where an increased incidence of EBV infections was not observed.

### REFERENCE

Cesarman E (2014). Gammaherpesviruses and lymphoproliferative disorders. Annu Rev Pathol, 9(349-372).
Cohen J I (2015). Primary Immunodeficiencies Associated with EBV Disease. Curr Top Microbiol Immunol, 390(Pt 1):241-265.
Cordoba, F., Wieczorek, G., Audet, M. et al. (2015). A novel, blocking, Fc-silent anti-CD40 monoclonal antibody prolongs nonhuman primate renal allograft survival in the absence of B cell depletion. Am J Transpl, 15, 2825-2836.
Larsen C P, Pearson T C, Adams A B et al. (2005). Rational development of LEA29Y (belatacept), a high-affinity variant of CTLA4-Ig with potent immunosuppressive properties. Am J Trans, 5(3):443-453.
Ristov J, Espie P, Ulrich P et al. (2018). Characterization of the in vitro and in vivo properties of CFZ533, a blocking and non-depleting anti-CD40 monoclonal antibody. Am J Transplant, 18(12):2895-2904.
Vincenti F, Larsen C, Durrbach A et al. (2005). Costimulation blockade with belatacept in renal transplantation. NEJM, 353(8):770-781.
Ma S D, Xu X, Plowshay J et al. (2015). LMP1-deficient Epstein-Barr virus mutant requires T cells for lymphomagenesis. J Clin Invest, 125(1):304-315.
Andorsky D J, Yamada R E, Said J et al. (2011) Programmed death ligand 1 is expressed by non-hodgkin lymphomas and inhibits the activity of tumor-associated T cells. Clin Cancer Res, July 1; 17(13):4232-4244.

1. A method of preventing an EBV-associated disorder in a subject, comprising administering to said subject a therapeutically effective dose of a CD40 antagonist.

2. A method according to claim 1, wherein the subject is a pediatric patient.

3. The method according to claim 1 or 2, wherein said subject will undergo an organ or tissue transplant.

4. A method of according to claim 3, wherein the subject is an EBV-seronegative transplant patient who receives an organ from an EBV seropositive donor.

5. A method according to claim 3, wherein the patient is a liver transplants patient.

6. A method according to claim 3, wherein said subject is immunosuppressed.

7. A method according to claim 3, wherein the EBV-associated disorder is cancer or a lymphoproliferative disease.

8. A method of reducing the likelihood that a subject will develop an EBV-associated disorder, comprising administering to the subject a therapeutically effective dose of a CD40 antagonist.

9. A method according to claim 8, wherein the patient is a pediatric patient.

10. The method according to claim 8 or 9, wherein said subject will undergo an organ or tissue transplant.

11. A method of according to claim 10, wherein the subject is an EBV seronegative transplant patient who receives an organ from an EBV positive donor.

12. A method according to claim 10, wherein the patient is a liver transplants patient.

13. A method according to claim 10, wherein said subject is immunosuppressed.

14. A method according to claim 10, wherein the EBV-associated disorder is cancer or a lymphoproliferative disease.

15. The method according to claim 14, wherein said method reduces the likelihood of post-transplant lymphoproliferative disease.

16. A method of transplanting a solid organ to patient in need thereof, comprising administering to the subject a CD40 antagonist.

17. A method according to claim 16, wherein the patient is a pediatric patient.

18. A method of according to claim 16 or 17, wherein the subject is an EBV seronegative transplant patient who receives an organ from an EBV positive donor.

19. A method according to claim 18, wherein the patient is a liver transplants patient.

20. A method according to claim 18, wherein said subject is immunosuppressed.

21. A method of controlling EBV infection in a subject, comprising administering to said subject a therapeutically effective dose of a CD40 antagonist.

22. A method according to claim 21, wherein the patient is a pediatric patient.

23. The method according to claims 21 or 22, wherein said subject will undergo an organ or tissue transplant.

24. A method of according to claim 23, wherein the subject is an EBV seronegative transplant patient who receives an organ from an EBV positive donor.

25. A method according to claim 24, wherein the patient is a liver transplants patient.

26. A method according to claim 24, wherein said subject is immunosuppressed.

27. A method according to the claims 1, 8, 16 or 21, wherein the CD40 antagonist is an anti-CD40 antibody.

28. A method according to claim 27, wherein the CD40 antagonist is selected from a group of anti-CD40 antibodies consisting of ASKP1240, BI655064 and FFP104.

29. A method according claim 27, wherein said antibody is anti-CD40 antibody with silenced ADCC activity.

30. A method according to claim 27, wherein the antibody is selected from the group consisting of:
e. an anti-CD40 antibody comprising an immunoglobulin VH domain comprising the amino acid sequence of SEQ ID NO: 7 and an immunoglobulin VL domain comprising the amino acid sequence of SEQ ID NO: 8;
f. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
g. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13; and
h. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14.

31. A method according to claims 28 or 30, wherein the antibody comprises the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

32. A method according to claim 27, wherein the antibody is CFZ533.

33. A method according to claim 32, wherein the antibody CFZ533 is used in combination with CsA, tacrolimus or mTor inhibitor such as everolimus.


SEQUENCE LISTING (19 sequences)

SEQ ID NO: 1 - protein, 5 aa, Homo sapiens
SYGMH

SEQ ID NO: 2 - protein, 17 aa, Homo sapiens
VISYEESNRYHADSVKG

SEQ ID NO: 3 - protein, 11 aa, Homo Sapiens
DGGIAAPGPDY

SEQ ID NO: 4 - protein, 16 aa, Homo sapiens
RSSQSLLYSNGYNYLD

SEQ ID NO: 5 - protein, 7 aa, Homo sapiens
LGSNRAS

SEQ ID NO: 6 - protein, 9 aa, Homo sapiens
MQARQTPFT

SEQ ID NO: 7 - protein, 120 aa, Homo sapiens
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSS

SEQ ID NO: 8 - protein, 112 aa, Homo sapeins
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIR

SEQ ID NO: 9 - protein, 450 aa, Homo sapiens
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, Homo sapiens
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 450 aa, Homo sapiens
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 219 aa, Homo sapiens; identical to SEQ ID NO: 10

SEQ ID NO: 13 - protein, 217 aa, Homo sapiens
APELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 217 aa, Homo sapiens
APELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1350 bp, Homo sapiens
caggtgcagctggtggaatctggcggcggagtggtgcagcctggccggtccctgagactgtcttgcgccgcctccggcttcaccttctccagctacggcatgcactgggtgcgacaggcccctggcaagggactggaatgggtggccgtgatctcctacgaggaatccaacagataccacgctgactccgtgaagggccggttcacaatctcccgggacaactccaagatcaccctgtacctgcagatgaactccctgcggaccgaggacaccgccgtgtactactgcgccagggacggaggaatcgccgctcctggacctgattattggggccagggcaccctggtgacagtgtcctccgctagcaccaagggcccctccgtgttccctctggccccctccagcaagtccacctctggcggcaccgccgctctgggctgcctggtgaaagactacttccccgagcccgtgaccgtgtcctggaactctggcgccctgacctccggcgtgcacacctttccagccgtgctgcagtcctccggcctgtactccctgtcctccgtggtgaccgtgccctctagctctctgggcacccagacctacatctgcaacgtgaaccacaagccctccaacaccaaggtggacaagcgggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacgcctccacctaccgggtggtgtctgtgctgaccgtgctgcaccaggactggctgaacggcaaagagtacaagtgcaaggtctccaacaaggccctgcctgcccccatcgaaaagaccatctccaaggccaagggccagccccgcgagccacaggtgtacacactgccccccagccgggaagagatgaccaagaaccaggtgtccctgacctgtctggtcaaaggcttctacccctccgatatcgccgtggagtgggagtccaacggacagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

SEQ ID NO: 16 - DNA, 657 bp, Homo sapiens
gacatcgtgatgacccagtcccccctgtccctgaccgtgacacctggcgagcctgcctctatctcctgcagatcctcccagtccctgctgtactccaacggctacaactacctggactggtatctgcagaagcccggccagtccccacaggtgctgatctccctgggctccaacagagcctctggcgtgcccgaccggttctccggctctggctctggcaccgacttcacactgaagatctcacgggtggaagccgaggacgtgggcgtgtactactgcatgcaggcccggcagacccccttcaccttcggccctggcaccaaggtggacatccggcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 17 - DNA, 1350 bp, Homo sapiens
caggtgcagctggtggaatctggcggcggagtggtgcagcctggccggtccctgagactgtcttgcgccgcctccggcttcaccttctccagctacggcatgcactgggtgcgacaggcccctggcaagggactggaatgggtggccgtgatctcctacgaggaatccaacagataccacgctgactccgtgaagggccggttcacaatctcccgggacaactccaagatcaccctgtacctgcagatgaactccctgcggaccgaggacaccgccgtgtactactgcgccagggacggaggaatcgccgctcctggacctgattattggggccagggcaccctggtgacagtgtcctccgctagcaccaagggcccctccgtgttccctctggccccctccagcaagtccacctctggcggcaccgccgctctgggctgcctggtgaaagactacttccccgagcccgtgaccgtgtcctggaactctggcgccctgacctccggcgtgcacacctttccagccgtgctgcagtcctccggcctgtactccctgtcctccgtggtgaccgtgccctctagctctctgggcacccagacctacatctgcaacgtgaaccacaagccctccaacaccaaggtggacaagcgggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggccgtgtcccacgaggaccctgaagtgaagttcaattggtacgtggacggcgtggaagtgcacaacgccaagaccaagcccagagaggaacagtacaactccacctaccgggtggtgtctgtgctgaccgtgctgcaccaggactggctgaacggcaaagagtacaagtgcaaggtctccaacaaggccctgcctgcccccatcgaaaagaccatctccaaggccaagggccagccccgcgagccacaggtgtacacactgccccccagccgggaagagatgaccaagaaccaggtgtccctgacctgtctggtcaaaggcttctacccctccgatatcgccgtggagtgggagtccaacggacagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

SEQ ID NO: 18 - DNA, 657 bp, Homo sapiens; identical to SEQ ID NO: 16

SEQ ID NO: 19 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ